SEQUENCE LISTING (48 sequences)

SEQ ID NO: 1 - DNA, 160 bp, Bacteriophage T7
ttctagagcacagctaacaccacgtcgtccctatctgctgccctaggtctatgagtggttgctggataactttacgggcatgcataaggctcgtatgatatattcagggagaccacaacggtttccctctacaaataattttgtttaacttttactagag

SEQ ID NO: 2 - DNA, 4107 bp, Streptococcus pyogenes
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagactaa

SEQ ID NO: 3 - RNA, 12 nt, Artificial Sequence
  Synthetic Linker
ggugguggagga

SEQ ID NO: 4 - DNA, 243 bp, Aequorea victoria
aagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 5 - DNA, 474 bp, Aequorea victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcag

SEQ ID NO: 6 - DNA, 54 bp, Streptococcus pyogenes
ctgataaatttctttgaatttctccttgattatttgttataaatgttataaaat

SEQ ID NO: 7 - DNA, 20 bp, Artificial Sequence
  Target Sequence
tgaaccaacgcatgacccaa

SEQ ID NO: 8 - DNA, 20 bp, Artificial Sequence
  Target Sequence
ggaaagatgctatcttccga

SEQ ID NO: 9 - DNA, 89 bp, Streptococcus pyogenes
gttggaaccattcaaaacagcatagcaagttaaaataaggctagtccgttatcaacttgaaaaagtggcaccgagtcggtgcttttttt

SEQ ID NO: 10 - DNA, 42 bp, Streptococcus pyogenes
taaaaatgataaaacaagcgttttgaaagcgcttgttttttt

SEQ ID NO: 11 - DNA, 69 bp, Artificial Sequence
  J23100 Constitutive Promoter
gacaatgaaaacgttagtcatggcgcgccttgacggctagctcagtcctaggtacagtgctagcttaat

SEQ ID NO: 12 - DNA, 739 bp, Artificial Sequence
  pBR322 Origin of Replication
gatcaaaggatcttcttgagatcctttttttctgcgcgtaatcttttgccctgtaaacgaaaaaaccacctggggaggtggtttgatcgaaggttaagtcagttggggaactgcttaaccgtggtaactggctttcgcagagcacagcaaccaaatctgtccttccagtgtagccggactttggcgcacacttcaagagcaaccgcgtgtttagctaaacaaatcctctgcgaactcccagttaccaatggctgctgccagtggcgttttaccgtgcttttccgggttggactcaagtgaacagttaccggataaggcgcagcagtcgggctgaacggggagttcttgcttacagcccagcttggagcgaacgacctacaccgagccgagataccagtgtgtgagctatgagaaagcgccacacttcccgtaagggagaaaggcggaacaggtatccggtaaacggcagggtcggaacaggagagcgcaagagggagcgacccgccggaaacggtggggatctttaagtcctgtcgggtttcgcccgtactgtcagattcatggttgagcctcacggctcccacagatgcaccggaaaagcgtctgtttatgtgaactctggcaggagggcggagcctatggaaaaacgccaccggcgcggccctgctgttttgcctcacatgttagtcccctgcttatccacggaatctgtgggtaactttgtatgtgtccgcagcgc

SEQ ID NO: 13 - DNA, 789 bp, Escherichia coli
atgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaa

SEQ ID NO: 14 - protein, 4 aa, Artificial Sequence
  Peptide Linker
GGGG

SEQ ID NO: 15 - protein, 5 aa, Artificial Sequence
  Peptide Linker
GGGGS

SEQ ID NO: 16 - DNA, 49 bp, Streptococcus thermophilus
agctgtagggaaactaaaagagaaatattggaagcaagccatagcagaa

SEQ ID NO: 17 - DNA, 69 bp, Streptococcus thermophilus
tattggaagcaagccatagcagaatatgaaaaacgtttaggcccatacaccaagatagacatcatagaa

SEQ ID NO: 18 - DNA, 66 bp, Streptococcus thermophilus
tacaccaagatagacatcatagaagttccagacgaaaaagcaccagaaaatatgagcgacaaagaa

SEQ ID NO: 19 - DNA, 48 bp, Streptococcus thermophilus
ccagaaaatatgagcgacaaagaaattgagcaagtaaaagaaaaagaa

SEQ ID NO: 20 - DNA, 50 bp, Streptococcus thermophilus
ttgaaccaacgcatgacccaagggcaaagcgactttgtattcgtcattgg

SEQ ID NO: 21 - DNA, 59 bp, Streptococcus thermophilus
ggaaagatgctatcttccgaaggattggcccaagagttgaaccaacgcatgacccaagg

SEQ ID NO: 22 - DNA, 52 bp, Streptococcus thermophilus
tgaaccaacgcatgacccaagggcaaagcgactttgtattcgtcattggcgg

SEQ ID NO: 23 - DNA, 60 bp, Streptococcus thermophilus
ggaaagatgctatcttccgaaggattggcccaagagttgaaccaacgcatgacccaaggg

SEQ ID NO: 24 - DNA, 49 bp, Streptococcus thermophilus
gtcattacattagaaatacaaggaaagatgctatcttccgaaggattgg

SEQ ID NO: 25 - DNA, 55 bp, Streptococcus thermophilus
gatgctatcttccgaaggattggcccaagagttgaaccaacgcatgacccaaggg

SEQ ID NO: 26 - DNA, 1560 bp, Trypanosoma brucei
aactgcaaaaaatattggtataataagagggaacagtgtgaacaagttaataacttgtggataactggaaagttgataacaatttggaggaccaaacgacatgaaaatcaccattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatccacagtcattacattagaaatacaaggaaagatgctatcttccgaaggattggcccaagagttgaaccaacgcatgacccaagggcaaagcgactttgtattcgtcattggcggatcaaacggcctgcacaaggatgtcttacaacgcagtaactacgcactatcattcagcaaaatgacatttccacaccaaatgatgcgggttgtgttaattgagcaagtgtatagagcatttaagattatgcgtggagaagcgtaccacaaataaaactaaaaaatagattgcgtagcacatattatgaaataattcattagataaaggagaaattgttaatgactatgtttcgtgaggcattaatatggctagtactcctagtatttaatttaataaacacgttcttagttattataggggggaaaacacaattatttaaagttccactatggagtacgtggctattatgaattattacgatcattatactaggtattttattctttagaaaatatctacaaaaaacgtattctctaactaatataaattccgataaaaagtttaaagacggtgagttctttgtacaaatccctttatacatcattgagaatcaaagcaatgttatatacggtaacgagacaataacgtataaacctgtttttgttaatatatttcataaattattgagtctctatggtgttcaaacaaaatatagtgtatatatgaattctagagagaacaatgtaaaagtaattcgtaaacatgtggtagcgaataaacatcaatatacgatgtatttgaatgatgaagaagaaggcatacttgagatgaaacagttcttcaaaagtgggggaaagcaacaaattccttatacgtttaattacaaatctgagttatttgatgtaagcaatccgttttttagtaatgaaaccaaaattacatttgagaatgaagtattattaaccgcaaagcgtagttttttagatatttcaaaaagtaaactgactaaaaaacgtggggaaaaacacaatatacacattcacagtactagagtagagaaagaaatattaatagccatttacttacaatgcatgataaacaagcaaacacaataaatgaagtataggtgtagtataaatgaatcaaaataataattgatttaaccattaacgaataaagattttagtacaaatataccctattatcataactgctaaaaaagatagtgaaggcaacaaaacaaaccatattga

SEQ ID NO: 27 - DNA, 1091 bp, Trypanosoma brucei
caccattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatccacagtcattacattagaaatacaaggaaagatgctatcttccgaaggattggcccaagagttgaaccaacgcatgacccaagggcaaagcgactttgtattcgtcattggcggatcaccattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatccacagtcattacattagaaatacaaggaaagatgctatcttccgaaggattggcccaagagttgaaccaacgcatgacccaagggcaaagcgactttgtattcgtcattggcggatcaccattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatccacagtcattacattagaaatacaaggaaagatgctatcttccgaaggattggcccaagagttgaaccaacgcatgacccaagggcaaagcgactttgtattcgtcattggcggataatcaaaccacaatccacagtcattacattagaaatacaaggaaagatgctatcttccgaaggattggcccaagagttgaaccaacgcatgacccaagggcaaagcgactttgtattcgtcattggcggat

SEQ ID NO: 28 - DNA, 423 bp, Streptococcus pyogenes
  misc_feature (320)..(339): n is a, c, g, or t
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnngttttagagctagaaatagcaagttaaaataaggctagtccgttatcaacttgaaaaagtggcaccgagtcggtgctttttttt

SEQ ID NO: 29 - DNA, 471 bp, Streptococcus thermophilus
  misc_feature (320)..(339): n is a, c, g, or t
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnngtttttgtactctcaagatttaagtaactgtacaacgaaacttacacagttacttaaatcttgcagaagctacaaagataaggcttcatgccgaaatcaacaccctgtcattttatggcagggtgttttttt

SEQ ID NO: 30 - DNA, 429 bp, Streptococcus thermophilus
  misc_feature (320)..(339): n is a, c, g, or t
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnngtttttgtactctcagaaatgcagaagctacaaagataaggcttcatgccgaaatcaacaccctgtcattttatggcagggtgttttttt

SEQ ID NO: 31 - DNA, 483 bp, Neisseria meningitidis
  misc_feature (320)..(339): n is a, c, g, or t
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnngttgtagctccctttctcatttcgcagtgctacaatgaaaattgtcgcactgcgaaatgagaaccgttgctacaataaggccgtctgaaaagatgtgccgcaacgctctgccccttaaagcttctgctttaaggggcttttttt

SEQ ID NO: 32 - DNA, 447 bp, Neisseria meningitidis
  misc_feature (320)..(339): n is a, c, g, or t
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnngttgtagctccctttctcgaaagagaaccgttgctacaataaggccgtctgaaaagatgtgccgcaacgctctgccccttaaagcttctgctttaacgggcttttttt

SEQ ID NO: 33 - protein, 15 aa, Artificial Sequence
  Peptide Linker
GGGGGGGGGGGGGGG

SEQ ID NO: 34 - protein, 15 aa, Artificial Sequence
  Peptide Linker
GGGGSGGGGSGGGGS

SEQ ID NO: 35 - protein, 15 aa, Artificial Sequence
  Peptide Linker
GGGGSGGGGSGGGGT

SEQ ID NO: 36 - protein, 19 aa, Artificial Sequence
  Peptide Linker
LGGGGSGGGGSGGGGSAAA

SEQ ID NO: 37 - protein, 25 aa, Artificial Sequence
  Peptide Linker
LSGGGGSGGGGSGGGGSGGGGSAAA

SEQ ID NO: 38 - protein, 4 aa, Artificial Sequence
  Peptide Linker
LAAA

SEQ ID NO: 39 - protein, 5 aa, Artificial Sequence
  Peptide Linker
RSIAT

SEQ ID NO: 40 - protein, 17 aa, Artificial Sequence
  Peptide Linker
RPACKIPNDLKQKVMNH

SEQ ID NO: 41 - protein, 17 aa, Artificial Sequence
  Peptide Linker
AAANSSIDLISVPVDSR

SEQ ID NO: 42 - protein, 15 aa, Artificial Sequence
  Peptide Linker
LQGGSGGGGSGGGGY

SEQ ID NO: 43 - protein, 17 aa, Artificial Sequence
  Peptide Linker
AEAAAKEAAAKEAAAKA

SEQ ID NO: 44 - protein, 15 aa, Artificial Sequence
  Peptide Linker
LAEAAAKEAAAKAAA

SEQ ID NO: 45 - protein, 20 aa, Artificial Sequence
  Peptide Linker
LAEAAAKEAAAKEAAAKAAA

SEQ ID NO: 46 - protein, 25 aa, Artificial Sequence
  Peptide Linker
LAEAAAKEAAAKEAAAKEAAAKAAA

SEQ ID NO: 47 - protein, 30 aa, Artificial Sequence
  Peptide Linker
LAEAAAKEAAAKEAAAKEAAAKEAAAKAAA

SEQ ID NO: 48 - protein, 55 aa, Artificial Sequence
  Peptide LInker
LFNKEQQNAFYEILHLPNLNEEQRNGFIQSLKDDPSQSANLLAEAKKLNDAQAAA

(19) United States
(12) Patent Application Publication
(10) Pub. No.: US 2015/0056629 A1
(43) Pub. Date: Feb. 26, 2015

(54) Compositions, systems, and methods for detecting a DNA sequence

(71) Applicant: Katriona Guthrie-Honea, Seattle, WA (US)

(72) Inventor: Katriona Guthrie-Honea, Seattle, WA (US)

(21) Appl. No.: 14/252,691

(22) Filed: Apr. 14, 2014

Related U.S. Application Data
(60) Provisional application No. 61/811,768, filed on Apr. 14, 2013.

(51) Int. Cl. C12Q 1/68; C07K 14/195; C12N 9/02

(52) U.S. Cl. CPC C12Q 1/6818; C12N 9/0069; C07K 14/195; C07K 2319/80; C07K 2319/60; C12Y 113/12007
USPC 435/6.19; 435/189; 536/23.4; 435/320.1

(57) ABSTRACT

Provided are compositions, systems, and methods that employ one or more fusion protein pairs, wherein each fusion protein within a fusion protein pair comprises a sequence-specific nucleic acid binding protein, such as sequence-specific Cas9 protein (e.g., a CRISPR), a sequence specific transcription activator-like enhancer (“TALE”) protein, a sequence specific homing endonuclease (“HE”; a/k/a meganuclease), a three prime exonuclease (“TREX”), and/or a sequence specific zinc finger (“ZF”) protein, which sequence-specific nucleic acid binding protein is operably linked to one half of a split-reporter molecule, such as a split-fluorescent reporter molecule, a split-luminescent reporter molecule, a Förster resonance energy transfer (FRET) reporter molecule, or a Bioluminescence Resonance Energy Transfer (BRET) reporter molecule.

### CROSS-REFERENCE TO RELATED APPLICATIONS

The present application was filed on Apr. 14, 2014 as a U.S. Non-provisional patent application and claims the benefit of U.S. Provisional Patent Application No. 61/811,768, filed Apr. 14, 2013, which provisional patent application is incorporated by reference herein in its entirety.

### BACKGROUND OF THE DISCLOSURE

1. Technical Field of the Disclosure

The present disclosure relates, generally, to the fields of genetic diagnostics and biosensors. More specifically, the present disclosure provides fusion proteins, as well as compositions, systems, and methods that employ such fusion proteins, for the detecting and/or identifying a nucleotide sequence, including a DNA sequence that is specific to a particular organism and/or that constitutes a DNA signature.

2. Description of the Related Art

High-specificity nucleic acid binding proteins, including Cas9 proteins, transcription activator-like enhancer (“TALE”) proteins, and homing endonucleases (“HE”) have been described as have methodologies for engineering variants of those nucleic acid binding proteins having a desired nucleotide sequence specificity.

CRISPRs (clustered regularly interspaced short palindromic repeats) are DNA loci that contain short nucleotide sequence repeats. Each repeat being followed by a short segment of “spacer DNA.” CRISPRs are often associated with cas genes, which encode CRISPR related proteins. The CRISPR/Cas system is believed to be a prokaryotic immune system that confers resistance to foreign genetic elements such as plasmids and phages; CRISPR spacers recognize and silence the exogenous genetic elements.

The CRISPR/Cas system has recently been exploited for the targeted silencing, enhancing, or alteration of specific genes eukaryotes including humans. A plasmid containing a cas gene and a specifically designed CRISPR can be engineered to generate a highly specific incision of a target sequence within an organism's genome.

Homing endonucleases comprise a broad range of endonucleases that catalyze the highly sequence-specific hydrolysis of genomic DNA within cells in which they are produced. Host-mediated repair of the hydrolyzed DNA often causes the gene encoding the homing endonuclease to become copied into the cleavage site—a process referred to as “homing.” The LAGLIDADG family of homing endonucleases has become valuable tools genome engineering. They can be used to replace, eliminate or modify sequences with a high degree of specificity. The target nucleic acid recognition sequence of a homing endonuclease can be modified through protein engineering and can be used to modify all genome types, whether bacterial, plant, or animal.

Transcription activator-like effector nucleases (TALENs) are artificial restriction enzymes generated by fusing a TAL effector DNA binding domain to a DNA cleavage domain. Because of the modularity of the DNA binding domain, transcription activator-like effectors (TALEs) can be engineered to bind to a desired DNA sequence. By combining such an engineered TALE with a DNA cleavage domain, highly sequence specific restriction enzymes have been produced that can be used genome editing in situ. TALEs comprise one or more highly conserved repeat domains, each of which binds to a single base pair of DNA.

The identities of two residues (referred to as repeat variable di-residues or RVDs) in these 33 to 35 amino acid repeats are associated with the binding specificity of these domains. TAL effector repeats can be joined together to create extended arrays, which are capable of binding to target DNA sequences of interest. Efficient DNA-binding by TAL effector repeat arrays also requires the presence of additional N-terminal and C-terminal amino acid sequences derived from naturally occurring TAL effectors. A variety of assembly platforms have been developed that permit the assembly of DNA encoding customized TAL effector repeat arrays. Engineered TAL repeat arrays can be fused to functional domains to create artificial proteins with novel functions. Repair of double-strand DNA breaks induced by TALENs has been exploited to induce targeted insertion/deletion mutations (by non-homologous end-joining-mediated repair) or specific substitutions or insertions (by homology-directed repair). TAL effector repeat arrays have also been fused to transcriptional regulatory domains to create artificial transcription factors.

The ability of certain proteins to be divided into independent and functional domains is well known. Such “split proteins” include dihydrofolate reductase (DHFR), beta-lactamase, yeast Ga14, tobacco etch virus protease, ubiquitin, and LacZ. More recently split reporter proteins, such as split luciferase and split green fluorescent protein have been described. The most common split reporters include firefly luciferase, renilla luciferase, green fluorescent protein (GFP) and its variants with various spectral properties, which have been exploited to study protein-protein interactions, protein localization, intracellular protein dynamics, and protein activity in living cells and animals.

### SUMMARY OF THE DISCLOSURE

The present disclosure provides, inter alia, fusion proteins, in particular fusion protein pairs, as well as compositions, systems, and methods that employ such fusion protein pairs for the detection of a target nucleic acid sequence. The fusion proteins disclosed herein comprise a sequence specific nucleic acid targeting protein in operable combination with (i.e., linked to) at least a portion of a reporter molecule, such as a split-reporter molecule.

Within certain embodiments, the presently disclosed compositions, systems, and methods employ one or more fusion protein pairs, wherein each fusion protein within a fusion protein pair comprises a sequence-specific nucleic acid binding protein, such as sequence-specific Cas9 protein (e.g., a CRISPR), a sequence specific transcription activator-like enhancer (“TALE”) protein, a sequence specific homing endonuclease (“HE”; a/k/a meganuclease), and/or a sequence specific zinc finger (“ZF”) protein, which sequence-specific nucleic acid binding protein is operably linked to one half of a split-reporter molecule, such as a split-fluorescent reporter molecule, a split-luminescent reporter molecule, a Förster resonance energy transfer (FRET) reporter molecule, or a Bioluminescence Resonance Energy Transfer (BRET) reporter molecule.

Also provided herein are polynucleotides that encode one or more fusion protein(s), each fusion protein comprising a sequence-specific nucleic acid binding protein and at least a portion of a reporter molecule. Expression and delivery of these polynucleotides may be achieved by employing a vector, such as a plasmid vector or a viral vector, such as a cocal vesiculovirus pseudotyped lentiviral vector, a foamy virus vector, an adenoviral vector, or an adeno-associated viral (AAV) vector.

The present disclosure also provides systems for detecting a target nucleic acid, which comprises two target nucleotide sequences, which systems comprise a first fusion protein and a second fusion protein, the first fusion protein comprising a first nucleotide sequence specific targeting protein in operable combination with a first portion of a split-reporter molecule and the second fusion protein comprising a second nucleotide sequence specific targeting protein in operable combination with a second portion of a split-reporter molecule, wherein the first nucleotide sequence specific targeting protein binds to a first target nucleotide sequence and the second nucleotide sequence specific targeting protein binds to a second target nucleotide sequence and wherein when the first and second target nucleotide sequences are in proximity the binding of the first fusion protein to the first target nucleotide sequence and the binding of the second fusion protein to the second target nucleotide sequence brings the first portion of the split-reporter molecule into juxtaposition with the second portion of the split-reporter molecule thereby restoring the functionality of the re-assembled split-reporter molecule and facilitating the detection of the target nucleic acid.

Within certain aspects of these embodiments, the first and second fusion proteins comprise first and second Transcription Activator-like (“TAL”) effector proteins having specificity for the first and second target nucleotide sequences, respectively. Within other aspects of these embodiments, the first and second fusion proteins comprise first and second homing endonucleases “HEs”) having specificity for the first and second target nucleotide sequences, respectively. Within further aspects of these embodiments, the first and second fusion proteins comprise a Cas protein, such as a Cas9 protein, and a tracrRNA having specificity for the first and second target nucleotide sequences, respectively. Within still further aspects of these embodiments, the first and second fusion proteins comprise first and second three prime repair endonucleases (“TREX”) having specificity for the first and second target nucleotide sequences, respectively. Within certain aspects of these embodiments, the first and second fusion proteins comprise first and second zinc finger (“ZF”) proteins having specificity for the first and second target nucleotide sequences, respectively.

Within related aspects of these embodiments, the first and second fusion proteins comprise first and second reporter molecules that are selected from split-fluorescent reporter molecules, split-luminescent reporter molecules, Förster resonance energy transfer (FRET) reporter molecules, and Bioluminescence Resonance Energy Transfer (BRET) reporter molecules.

Within other embodiments, the present disclosure provides methods that employ the contacting of a first fusion protein and a second fusion protein to a sample comprising a nucleic acid, wherein the first fusion protein comprises a first sequence specific nucleic acid binding protein in operable combination with a first portion of a split-reporter molecule and the second fusion protein comprises a second sequence specific nucleic acid binding protein in operable combination with a second portion of the split-reporter molecule, wherein the first sequence specific nucleic acid binding protein binds to a first target nucleotide sequence and the second sequence specific nucleic acid binding protein binds to a second target nucleotide sequence and wherein when the first and second nucleotide sequences are both present within the nucleic acid within sample and are both in proximity, the binding of the first sequence specific nucleic acid binding protein to the first target nucleotide sequence and the binding of the second gene-targeting protein to the second target nucleotide sequence brings the first portion of the reporter molecule into juxtaposition with the second portion of the reporter molecule thereby restoring the functionality of the re-assembled split-reporter molecule and facilitating the detection of the target nucleic acid.

Within certain aspects of these embodiments, the nucleic acid sample is contacted with first and second fusion proteins, which comprise first and second sequence specific nucleic acid binding proteins, respectively, that are Transcription Activator-like (TAL) effector proteins. Within other aspects of these embodiments, the nucleic acid sample is contacted with first and second fusion proteins, which comprise first and second sequence specific nucleic acid binding proteins, respectively, that are homing endonucleases (“HEs”) having specificity for the first and second target nucleotide sequences, respectively. Within other aspects of these embodiments, the nucleic acid sample is contacted with first and second fusion proteins, which comprise a Cas protein, such as a Cas9 protein, and a tracrRNA having specificity for the first and second target nucleotide sequences, respectively. Within other aspects of these embodiments, the nucleic acid sample is contacted with first and second fusion proteins, which comprise first and second sequence specific nucleic acid binding proteins, respectively, that are three prime repair endonucleases (“TREX”) having specificity for the first and second target nucleotide sequences, respectively. Within other aspects of these embodiments, the nucleic acid sample is contacted with first and second fusion proteins, which comprise first and second sequence specific nucleic acid binding proteins, respectively, that are zinc finger (“ZF”) proteins having specificity for the first and second target nucleotide sequences, respectively.

Within related aspects of these embodiments, the first and second fusion proteins comprise first and second reporter molecules that are selected from split-fluorescent reporter molecules, split-luminescent reporter molecules, Förster resonance energy transfer (FRET) reporter molecules, and Bioluminescence Resonance Energy Transfer (BRET) reporter molecules.

### BRIEF DESCRIPTION OF THE DRAWINGS

Certain aspects of the present disclosure will be better understood in view of the following figures:

FIG. 1 is a diagrammatic representation of an exemplary system for the detection and identification of a nucleic acid sequence using a sequence-specific nucleic acid targeting protein and as a split-reporter protein, the split-Renilla reniformis luciferase reporter protein).

FIG. 2 is a diagrammatic representation of an exemplary system for the genetic identification of a genetic sequence using Förster resonance energy transfer (FRET).

FIG. 3 is a hairpin structure of S. pyogenes Cas9 guide RNA gRNA-SPm

FIG. 4 is a hairpin structure of S. thermophilus Cas9 guide RNA gRNA-ST1f1

FIG. 5 is a hairpin structure of S. thermophilus Cas9 guide RNA gRNA-ST1m1

FIG. 6 is a hairpin structure of N. meningitidis Cas9 guide RNA gRNA-NMf

FIG. 7 is a hairpin structure of N. meningitidis Cas9 guide RNA gRNA-NM1

### DETAILED DESCRIPTION OF THE DISCLOSURE

The present disclosure is directed, generally, to fusion proteins, in particular fusion protein pairs, and compositions, systems, and methods employing fusion protein pairs for detecting a target nucleic acid sequence, including a target DNA or RNA sequence, such as a target nucleic acid sequence that is specific for a particular cell or organism and/or that constitutes at least a portion of a genetic signature, such as a DNA or RNA signature.

Within certain aspects, the presently disclosed compositions, systems, and methods employ fusion proteins or nucleic acids that encode fusion proteins, wherein each fusion protein of a fusion protein pair comprises a sequence-specific nucleic acid (e.g., DNA or RNA) targeting protein in operable combination with one half of a split-reporter molecule, such as a split-reporter protein including, e.g., a split luminescence protein, a split fluorescence protein, a split enzymatic protein, or other split protein.

It will be understood that, unless indicated to the contrary, terms intended to be “open” (e.g., the term “including” should be interpreted as “including but not limited to,” the term “having” should be interpreted as “having at least,” the term “includes” should be interpreted as “includes but is not limited to,” etc.). Phrases such as “at least one,” and “one or more,” and terms such as “a” or “an” include both the singular and the plural.

It will be further understood that where features or aspects of the disclosure are described in terms of Markush groups, the disclosure is also intended to be described in terms of any individual member or subgroup of members of the Markush group. Similarly, all ranges disclosed herein also encompass all possible sub-ranges and combinations of sub-ranges and that language such as “between,” “up to,” “at least,” “greater than,” “less than,” and the like include the number recited in the range and includes each individual member.

All references cited herein, whether supra or infra, including, but not limited to, patents, patent applications, and patent publications, whether U.S., PCT, or non-U.S. foreign, and all technical and/or scientific publications are hereby incorporated by reference in their entirety.

While various embodiments have been disclosed herein, other embodiments will be apparent to those skilled in the art. The various embodiments disclosed herein are for purposes of illustration and are not intended to be limiting, with the true scope and spirit being indicated by the claims.

### Nucleic Acid Binding Proteins for Achieving High-Specificity Binding to a Target Nucleic Acid Sequence

As discussed herein, the present disclosure provides fusion proteins, in particular fusion protein pairs, as well as compositions, systems, and methods that employ one or more fusion protein pairs wherein each fusion protein comprises a target sequence specific nucleic acid binding protein and a split-reporter protein, which fusion protein pairs permit the highly-specific detection of a DNA sequence.

Exemplified herein are fusion proteins comprising a sequence-specific nucleic acid binding proteins, such as sequence-specific Cas9 proteins (e.g., CRISPRs), sequence specific transcription activator-like enhancer (“TALE”) proteins, sequence specific homing endonucleases (“HE”; a/k/a meganucleases), and sequence specific zinc finger (“ZF”) proteins, which are operably linked to one half of a split-reporter molecule, such as a split-fluorescent reporter molecule, a split-luminescent reporter molecule, a Förster resonance energy transfer (FRET) reporter molecule, or a Bioluminescence Resonance Energy Transfer (BRET) reporter molecule.

It will be understood that the fusion proteins disclosed herein are intended for use in pairs wherein a first member of a pair of fusion proteins comprises a first sequence specific nucleic acid binding protein fused to a first half of a split-reporter molecule and a second member of a pair of fusion proteins comprises a second sequence specific nucleic acid binding protein fused to a second half of the split-reporter molecule.

Thus, as used in combination, a target nucleic acid is detected when a first fusion protein specifically binds to a first target sequence within the target nucleic acid and a second fusion protein specifically binds to a second target sequence within the target nucleic acid wherein binding of the first fusion protein and the second fusion protein to the target nucleic acid places the first half of a split-reporter molecule in juxtaposition with the second half of a split-reporter molecule such that the functionality of the reporter molecule is restored. Detection of the target nucleic acid, therefore, is achieved via the detection of a signal that results from the restored activity of the combined first and second halves of the reporter molecule.

As used herein, the term “sequence-specific nucleic acid targeting protein” refers, generally, to a class of proteins having a functional motif that associates with a nucleic acid in a sequence-specific manner. Such sequence-specific nucleic acid targeting proteins that may be employed in the fusion proteins disclosed herein include, for example, the three prime repair exonucleases (“TREX”), the finger nucleases (“ZFNs”), the transcriptional activator-like effectors (“TALEs”), the homing endonucleases (“HEs,” a/k/a meganucleases), and the clustered regularly interspersed short palindromic repeat proteins (“CRISPR”).

TALEs offer more straightforward modular design and higher DNA target specificity as compared to zinc finger nucleases. Homing endonucleases, such as LAGLIDADG homing endonucleases (LHEs), offer highly specific cleavage profiles and, because they are compact monomeric proteins that do not require dimerization as do ZFNs and TALEs, the ability to be used in multiplex combinations. Accordingly, HEs and CRISPRs (e.g., Cas9 in combination with an RNA guide strand) exhibit highly efficient, sequence specific target nucleic acid binding activity with minimal off-target effects. Mali et al., Science (2013), supra.

Specifically-designed nucleic acid targeting proteins may be tested for activity against a cognate target site and for off-target activity against any closely related genomic targets. TALEs, HEs, and Cas9 proteins may be engineered to avoid off-target genomic cleavage using the methods described in Stoddard, Structure 19:7-15 (2011) and Mali et al., Science (2013).

Three Prime Repair Exonucleases (“TREX”) Nucleic Acid Targeting Proteins

As used herein, the terms “three prime repair exonuclease” or “TREX” refer to non-processive 3′ to 5′ DNA exonucleases (e.g., “TREX1” and “TREX2”), which is typically involved in DNA replication, repair, and recombination. In humans, TREX exonucleases may serve a proofreading function for a DNA polymerase. TREX proteins are also components of the SET complex, which degrades 3′ ends of nicked DNA during granzyme A-mediated cell death. Mutations in this gene result in Aicardi-Goutieres syndrome, chilblain lupus, RVCL (Retinal Vasculopathy with Cerebral Leukodystrophy) and Cree encephalitis. Multiple transcript variants encoding different isoforms have been found for TREX1 and TREX2. Mazur and Perrino, J. Biol Chem 274(28):19655-60 (1999); Hoss et al., EMBO J 18(13):3868-75 (1999); and Crow et al., Nat Genet 38(8):917-20 (2006).

Transcription Activator-like Effector (“TALE”) Nucleic Acid Targeting Proteins

As used herein, the term “transcription activator-like effector,” “TAL effector,” and “TALE” refer to a class of highly specific DNA binding proteins that harbor highly conserved repeat domains that each bind to a single base pair of DNA. The identities of two residues (referred to as repeat variable di-residues or RVDs) in these 33 to 35 amino acid repeats are associated with the binding specificity of these domains.

Three assembly platforms have been described for achieving sequence-specific TAL effector proteins that may be suitably employed in the TAL effector fusion proteins described herein. Those assembly platforms include: (1) solid-phase methods; (2) standard cloning methods; and (3) Golden Gate assembly methods.

The solid phase assembly of DNA fragments encoding TAL effector repeat arrays using multi-channel pipets or automated liquid handling robots is described in Reyon et al., Nat. Biotechnol. 30:460-465 (2012); Briggs et al., Nucleic Acids Res. 40(15):e117 (2012); and Wang et al., Angew Chem. Int. Ed. Engl. 51(34):8505-8508 (2012).

The REAL methodology for the hierarchical assembly of DNA fragments encoding TAL effector repeat arrays using standard restriction digestion and ligation cloning methods is described in Sander et al., Nat. Biotechnol. (2011) and Huang et al. Nat. Biotechnol. (2011). “REAL-Fast” is a faster version of REAL, which follows the same assembly protocol as REAL but utilizes plasmids encoding pre-assembled TAL repeats rather than single TAL repeats. See, Reyon et al., Curr Protoc Mol Biol. (2012).

“Golden Gate” methods for assembling DNA encoding TAL effector repeat arrays, which methods are based on the simultaneous ligation of multiple DNA fragments encoding TAL repeat domains, are described by Cermak et al., Nucleic Acids Res. (2011); Li et al., Nucleic Acids Res. (2011); Morbitzer et al., Nucleic Acids Res. (2011); Weber et al., PLoS One (2011); Zhang et al., Nat. Biotechnol. (2011); and Li et al., Plant Mol. Biol. (2012).

The crystal structure of a TAL effector (PthXol) bound to its DNA target site has recently been determined. Mak et al., Science 335(6069):716-9 2012; e-pub 5 Jan. 2012 PubMed PMID: 22223736. These crystal structure data permit the precise definition of the boundaries of DNA recognition region and facilitates strategies for the creation of well-behaved TALE fusion constructs, which may be applied to achieve highly sequence specific nucleotide sequence detection. Specifically-designed TAL effector proteins can be tested for activity against a cognate target site and for off-target activity against any closely related genomic targets.

Homing Endonuclese Nucleic Acid Targeting Proteins

As used herein, the terms “homing endonuclease” and “meganuclease” refer to a class of restriction endonucleases that are characterized by recognition sequences that are long enough to occur only once in a genome and randomly with a very low probability (e.g., once every 7×10⁹ bp). Jasin, Trends Genet 12(6):224-8 (1996).

Each homing endonuclease belongs to one of the following six structural families, which are based primarily on conserved structural motifs (Belfort and Roberts Nucleic Acids Res 25(17): 3379-88 (1995)): (1) LAGLIDADG, (2) GIY-YIG, (3) His-Cys box, (4) H-N-H, (5) PD-(D/E)xK, and (6) Vsr-like.

LAGLIDADG homing endonucleases comprise one or two LAGLIDADG motifs, which is a conserved sequence that is directly involved in DNA cleavage. LAGLIDADG HEs are homodimers; each monomer interacts with the major groove of a DNA half-site. The LAGLIDADG motifs bind to both the protein-protein interface between individual HE subunits as well as to the enzyme's active site. HEs can be made to possess two LAGLIDADG motifs in a single protein chain, which permits the HE to act as a monomer.

The structures of the homing endonucleases PI-SceI and I-CreI were published by Heath et al. Nature Structural Biology 4(6):468-476 (1997) and Duan, Cell 89(4):555-564 (1997). The structure of I-CreI bound to its DNA target site is described in Jurica et al., Mol. Cell 1(4):469-76 (1998). The high-resolution crystal structures have recently been determined for ten separate LAGLIDADG HEs in complex with their cognate DNA target sites. Stoddard, Structure 19:7-15 (2011) and Takeuchi et al., Proc. Natl. Acad. Sci. U.S.A. 108:13077-13082 (2011).

Chimeric ‘hybrids’ of LAGLIDADG HEs have been constructed that provide a broad range of nucleic acid targeting proteins, which may be readily adapted for the sequence specific nucleic acid targeting proteins and fusion proteins of the present disclosure. Baxter et al., Nucl. Acids Res. 40(16):7985-8000 (2012).

GIY-YIG HEs have one GIY-YIG motif in the N-terminal region, which interacts with the DNA target sequence. GIY-YIG HEs are exemplified by the monomeric protein I-TevI. The structures of the I-TevI DNA-binding domain bound to a DNA target the I-TevI catalytic domain are described in Van Roey et al., Nature Structural Biology 9(11):806-811 (2002) and Van Roey et al., EMBO J 20(14):3631-3637 (2001).

His-Cys box HEs possess a 30 amino acid region that includes five conserved residues (two histidines and three cysteins), which co-ordinate a metal cation that is required for catalysis. I-PpoI is the best characterized HE within this family. The structure of the I-PpoI homodimer is described Flick et al., Nature 394(6688):96-101 (1998).

H-N-H HEs contain a 30 amino acid consensus sequence that includes two pairs of conserved histidines and one asparagine, which create a zinc finger nucleic acid binding domain. The structure of the monomeric I-HmuI HE is described in Shen et al., J Mol Biol 342(1):43-56 (2004).

PD-(D/E)xK HEs contain a canonical nuclease catalytic domain as is found in type II restriction endonucleases. The structure of the tetrameric I-Ssp6803I HE is described in Zhao et al., EMBO J 26(9):2432-2442 (2007).

Vsr-like HEs include a C-terminal nuclease domain having homology to the bacterial Very Short Patch Repair (Vsr) endonucleases. Vsr-like HEs are described in Dassa et al., Nucl Acids Res 37(8):2560-2573 (2009).

Two main approaches have been adopted to generate sequence specific nucleic acid targeting HEs that may be readily adapted for use in the fusion proteins disclosed herein. The specificity of existing HEs may be modified by introducing a small number of variations to the amino acid sequence within the nucleic acid binding domain. Functional HE variants having specificity for a target sequence of interest can be identified and isolated by the methodology described in tions of the natural recognition site. Seligman et al., Nucleic Acids Research 30(17):3870-9 (2002); Sussman et al., Journal of Molecular Biology 342(1):31-41 (2004); and Rosen et al., Nucl Acids Res 34(17):4791-800 (2006).

An alternative approach for generating target sequence specific HEs involves exploiting HEs' high degree of natural diversity via fusing domains from different molecules as is described in Arnould et al., J Mol Biol 355(3):443-58 (2006) and Smith et al., Nucl Acids Res 34(22):e149 (2006). This approach makes it possible to develop chimeric HEs with nes recognition sites that are composed of a half-site of a first HE and a half-site of a second HE. By, for example, fusing the protein domains of I-DmoI and I-CreI, the chimeric HEs E-DreI and DmoCre were created. Chevalier et al., Mol Cell 10(4):895-905 (2002).

Cellectis has developed a collection of over 20,000 protein domains from the homodimeric I-CreI HE as well as from other HE scaffolds. Grizot et al., Nucl Acids Res 38(6):2006-18. Precision Biosciences has developed a fully rational design process called Directed Nuclease Editor (DNE), which is capable of creating engineered HEs that bind to a user-defined target sequence. Gao et al., The Plant J 61(1):176-87 (2010). Bayer CropScience has described the application of DNE technology to precisely target a predetermined sequence for use in cotton plants, targeting it precisely to a predetermined site. Cotton, Bayer Research. These HEs can be further combined to generate functional chimeric HEs having a desired target sequence specificity and can, therefore, be adapted for use in the fusion proteins of the present disclosure.

HEs having suitable target sequence specificity may be identified by a yeast surface display strategy, combined with high-throughput cell sorting for desirable DNA cleavage specificity. A series of protein-DNA ‘modules’, which correspond to sequential pockets of contacts that extend across the entire target site, may be systematically randomized in separate libraries. Each library may then be systematically sorted for populations of enzymes that can specifically cleave each possible DNA variant within each module, and each sorted population deep-sequenced and archived for subsequent enzyme assembly and design. HEs that may be suitably employed in the compositions and methods of the present disclosure are commercially available (Pregenen, Seattle, Wash.).

Within certain aspects, the fusion proteins disclosed herein may comprise a target specific homing endonuclease variant such, for example, a target specific variant of a homing endonuclease selected from the group consisting of I-HjeMI, I-CpaMI, I-OnuI, I-CreI, PI-SceI, I-SceII, I-Dmol, I-TevI, I-TevII, I-TevIII, I-PpoI, I-PpolI, I-HmuI, I-HmuI, I-SSp68031, I-AniI, I-CeuI, I-ChuI, I-CpaI, I-CpaII, H-DreI, I-LlaI, I-MosI, PI-PfuI, PI-PkoII, I-PorI, PI-PspI, I-ScaI, I-SecIII, I-SceIV, I-SceV, I-SceVI, I-SceVII, PI-TLiI, PI-TLilI, I-Tsp061I, and I-Vdi141I.

CRISPR and Cas9 Nucleic Acid Targeting Proteins

As used herein, the terms “Clustered Regularly Interspaced Short Palindromic Repeats” and “CRISPR” refer to type II prokaryotic nucleic acid targeting proteins that were originally isolated from the bacterium Streptococcus pyogenes. CRISPR proteins having a small RNA strand that guides target nucleic acid sequence specificity thereby facilitating sequence-specific DNA binding.

As used herein, the terms “CRISPR/CRISPR-associated system” and “Cas” refer to endonucleases that uses an RNA guide strand to target the site of endonuclease cleavage. Thus, the term “CRISPR endonuclease” refers to a Cas endonuclease (e.g., the Cas9 endonuclease) in combination with an RNA guide strand. See, Jinek et al., Science 337:816-821 (2012); Cong et al., Science (Jan. 3, 2013) (Epub ahead of print); and Mali et al., Science (Jan. 3, 2013) (Epub ahead of print).

A CRISPR/CRISPR-associated system (Cas) includes a “spacer” for retention of foreign genetic material in clustered arrays within a host genome, a short guiding RNA (crRNA), which is encoded by a spacers, a protospacer that binds the crRNAs to a specific portion of the target DNA, and a CRISPR-associated nuclease (Cas) that degrades the protospacer.

In the bacterium Streptococcus pyogenes, four genes (Cas9, Cas1, Cas2, and CsnI) and two non-coding small RNAs (pre-crRNA and tracrRNA) act in concert to specifically bind to and degrade a target DNA. Jinek et. al. (2012), supra. The specificity of binding to target nucleic acid is controlled by non-repetitive spacer elements in the pre-crRNA that, in conjunction with the tracrRNA, directs the Cas9 nuclease to a protospacer:crRNA heteroduplex and induces the formation of a double-strand break (DSB).

Cas9 cleaves DNA only in the presence of a protospacer adjacent motif (PAM), which must be immediately downstream of the protospacer sequence. The PAM sequence, which in S. pyogenes comprises the canonical 5′-NGG-3′, wherein N refers to any nucleotide, and which can comprise the sequence NGG, NGGNG, NAAR, or NNAGAAW, is absolutely necessary for Cas9 binding and cleavage. Gasiunas et al., Proc Natl Acad Sci USA 109:E2579-2586 (2012); Xu et al., Appl Environ Microbio Epub (2014); Horvath and Barrangou, Science 327:167-170 (2010); van der Ploeg, Microbiology 155:1116-1121 (2009); and Deveau et al., J. Bacteriol. 190:1390-1400 (2008).

Expression of a single chimeric crRNA:tracrRNA transcript is sufficient for Cas9 sequence specificity. The endogenous S. pyogenes type II CRISPR/Cas system has been adapted for use in mammalian cells. It has been demonstrated that RNA-guided Cas9 can introduce precise double stranded breaks efficiently and with minimal off-target effects in mammalian cells. Cong et al. (2013); Mali et al. (2013); and Cho et al. (2013).

Several mutant forms of Cas9 nuclease have been developed to take advantage of their features for additional applications in genome engineering and transcriptional regulation. A tandem knockout of both the RuvCI and the HNH nuclease domains resulted in a Cas9 variant protein that is devoid of nuclease activity but retained binding specificity for a target nucleic acid sequence binding which exhibiting minimal off-binding. Qi et al., Cell 152(5):1173-83 (2013).

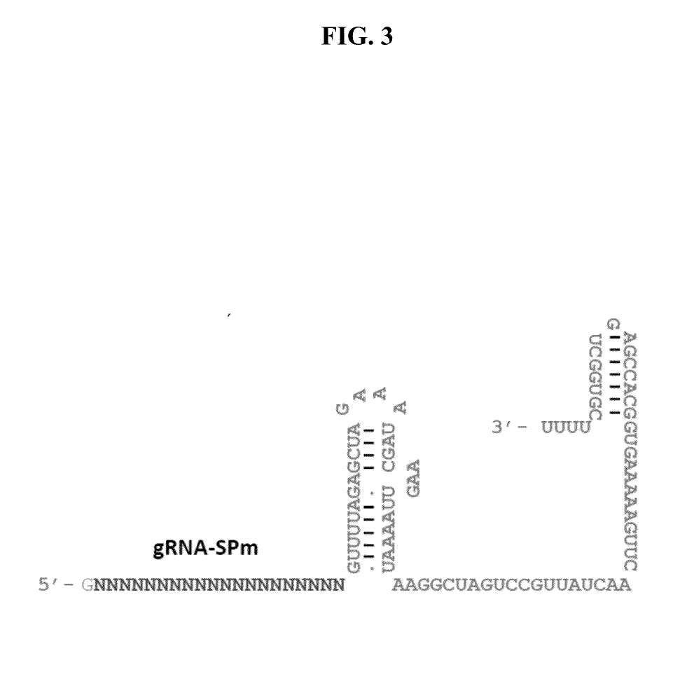

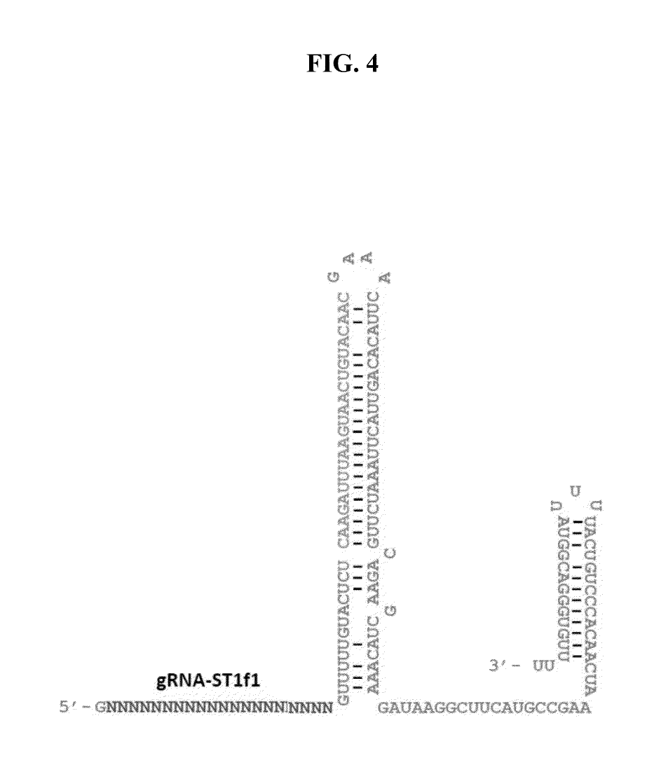

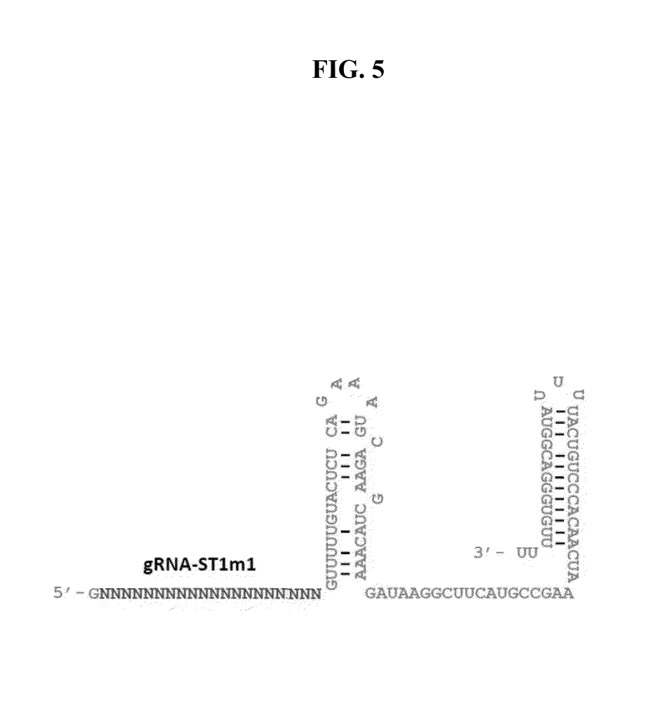

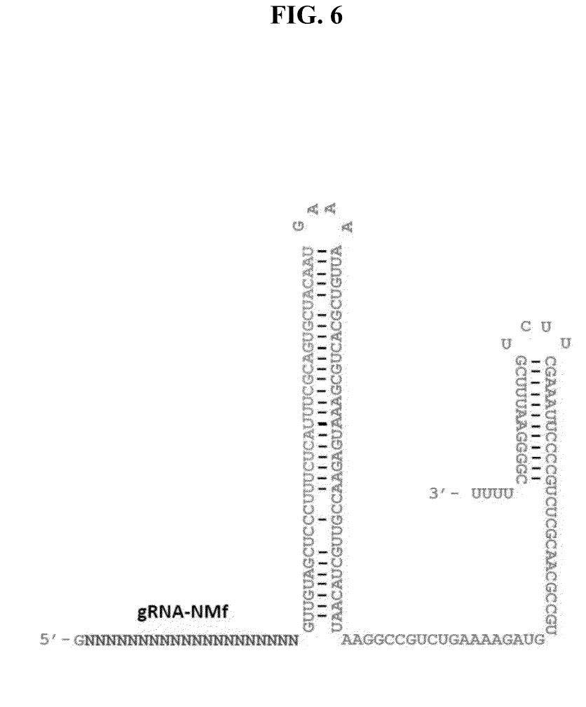

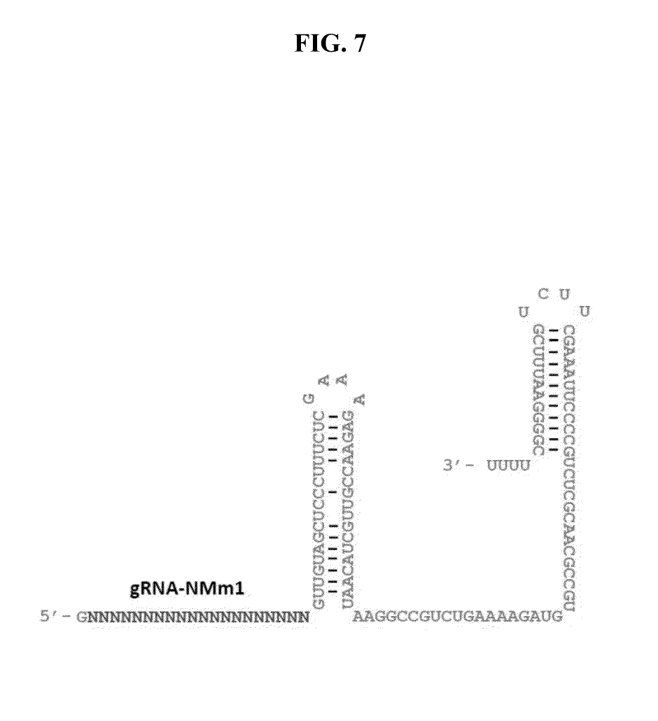

The CRISPR Type II RNA-guided endonuclease has two distinct components: (1) a guide RNA and (2) an endonuclease (i.e., the CRISPR associated (Cas) nuclease, Cas9). The guide RNA is a combination of the endogenous bacterial crRNA and tracrRNA in a single chimeric guide RNA (gRNA) transcript. The gRNA combines the targeting specificity of the crRNA with the scaffolding properties of the tracrRNA into a single transcript. When the gRNA and the Cas9 are expressed in the cell, the genomic target sequence can be modified or permanently disrupted. Exemplary gRNAs (showing secondary structure) for the Cas9-mediated detection of: S. pyogenes are presented in FIG. 3 and Table 1, SEQ ID NO: 28 (gRNA-SPm); S. thermophiles are presented in FIGS. 4-5 and Table 1, SEQ ID NOs: 29-30; and N. meningitides are presented in FIGS. 6-7 and Table 1, SEQ ID NOs: 31-32. Also presented in Table 1 are sequences of putative protospacer adjacent motif (PAM) sequences for S. thermophiles (SEQ ID NOs. 15-25); and nucleotide sequences of portions of the Ble antibiotic resistance gene (SEQ ID NOs: 26-27).

The gRNA/Cas9 complex is recruited to the target sequence by the base-pairing between the gRNA sequence and the complement to the target sequence in the genomic DNA. For successful binding of Cas9, the genomic target sequence must also contain the correct Protospacer Adjacent Motiff (PAM) sequence immediately following the target sequence. The binding of the gRNA/Cas9 complex localizes the Cas9 to the genomic target sequence so that the wild-type Cas9 can cut both strands of DNA causing a Double Strand Break (DSB). A DSB can be repaired through one of two general repair pathways: (1) the Non-Homologous End Joining (NHEJ) DNA repair pathway or (2) the Homology Directed Repair (HDR) pathway. The NHEJ repair pathway often results in inserts/deletions (InDels) at the DSB site that can lead to frameshifts and/or premature stop codons, effectively disrupting the open reading frame (ORF) of the targeted gene. The HDR pathway requires the presence of a repair template, which is used to fix the DSB. HDR faithfully copies the sequence of the repair template to the cut target sequence. Specific nucleotide changes can be introduced into a targeted gene by the use of HDR with a repair template.

TABLE 1
Sequence Elements for an Exemplary Cas9 Nuclease

| Sequence | | | | |
|---|---|---|---|---|
| Identifier | Sequence | Organism | Vector | Description |
| SEQ ID | agctgt [symbol] gaaactaaaagagaaatattggaagcaag | S. thermophilus | DS-ST1casN | Putative cas9 |
| NO: 16 | ccatagcagaa (1) | | | Targ Site |
| | | | | w/ PAM Seq |
| SEQ ID | tattggaagcaagccatagcagaatatgaaaaacgttt | S. thermophilus | DS-ST1casN | Putative cas9 |
| NO: 17 | [symbol] [symbol] cccatacaccaagatagacatcatagaa (21) | | | Targ Site |
| | | | | w/ PAM Seq |
| SEQ ID | tacaccaagatagacatcatagaagttccagacgaaaaag | S. thermophilus | DS-ST1casN | Putative cas9 |
| NO: 18 | caccagaaaatatgagcgacaaagaa (18) | | | Targ Site |
| | | | | w/ PAM Seq |
| SEQ ID | ccagaaaatatgagcgacaaagaaattgagcaagtaaaag | S. thermophilus | DS-ST1casN | Putative cas9 |
| NO: 19 | aaaa [symbol] (0) | | | Targ Site |
| | | | | w/ PAM Seq |
| SEQ ID | ttgaaccaacgcatgaccca [symbol] caaagcgactttgtat | S. thermophilus | DS-SPcasN | Putative cas9 |
| NO: 20 | tcgtcattgg(4) | | | Targ Site |
| | | | | w/ PAM Seq |
| SEQ ID | ggaaagatgctatcttccgaaggattggcccaagagttga | S. thermophilus | DS-SPcasN | Putative cas9 |
| NO: 21 | accaacgcatgacccaagg (13) | | | Targ Site |
| | | | | w/ PAM Seq |
| SEQ ID | tgaaccaacgcatgaccca [symbol] gactttgta | S. thermophilus | DS-SPcasN | Putative cas9 |
| NO: 22 | ttcgtcattggcgg (6) | | | Targ Site |
| | | | | w/ PAM Seq |
| SEQ ID | ggaaagatgctatcttccgaaaggattggcccaagagttg | S. thermophilus | DS-SPcasN | Putative cas9 |
| NO: 23 | aaccaacgcatgaccc [symbol] (14) | | | Targ Site |
| | | | | w/ PAM Seq |
| SEQ ID | gtcattacattagaaataca [symbol] ggaaagatgctatcttc | S. thermophilus | DS-SPcasN | Putative cas9 |
| NO: 24 | cg [symbol] attgg (3) | | | Targ Site |
| | | | | w/ PAM Seq |
| SEQ ID | gatgctatcttccgaaggattggcccaagagttgaaccaa | S. thermophilus | DS-SPcasN | Putative cas9 |
| NO: 25 | cgcatgacccaaggg (9) | | | |
| | | | | w/ PAM Seq |
| SEQ ID | aactgcaaaaaatattggtataataag [symbol] aacagtgt | | | Segment |
| NO: 26 | gaacaagttaataacttgtggataactggaaagttgataa | | | of Ble |
| | caatttggaggaccaaacgacatgaaaatcaccattttag | | | Antibiotic |
| | ctgt [symbol] gaaactaaaagagaaatattggaagcaagccat | | | Resistance |
| | agcagaatatgaaaaacgtttaggcccatacaccaagata | | | Gene |
| | gacatcatagaagttccagacgaaaaagcaccagaaaata | | | |
| | tgagcgacaaagaaattgagcaagt [symbol] aaaaga | | | |
| | [symbol] ccaacgaatactagccaaaatcaaaccacaatccacag | | | |
| | tcattacattagaaatacaaggaaagatgctatcttccga | | | |
| | [symbol] attggcccaagagttgaaccaacgcatgacccaaggg | | | |
| | caaagcgactttgtattcgtcat [symbol] cggatcaaacggc | | | |
| | ctgcacaaggatgtcttacaacgcagtaactacgcactat | | | |
| | cattcagcaaaatgacatttccacaccaaatgatgcgggt | | | |
| | tgtgttaattgagcaagtgtatagagcatttaagattat | | | |
| | gcgtg [symbol] [symbol] gcgtaccacaaataaaactaaaaaataga | | | |
| | ttgcgtagcacatattatgaaataattcattagataa | | | |
| | aggagaaattgttaatgactatgtttcgtgaggcattaata | | | |
| | tggctagtactcctagtatttaatttaataaacacgttc | | | |
| | ttagttattat [symbol] g [symbol] g [symbol] aaaacacaattatttaaa | | | |
| | gttccactatggagtacgtggctatta [symbol] gaattattac | | | |
| | gatcattatact [symbol] tattttattctttagaaaatatct | | | |
| | acaaaaaacgtattctctaactaatataaattccgataa | | | |
| | aaagtttaaagacggtgagttctttgtacaaatcccttta | | | |
| | tacatcattgagaatcaaagcaatgttatatacggtaacg | | | |
| | agacaataacgtataaaCctgtttttgttaatatatttca | | | |
| | taaattattgagtctctatggtgttcaaacaaaatatagt | | | |
| | gtatatatgaattctagagagaacaatgtaaaagtaattc | | | |
| | gtaaacatgtggtagcgaataaacatcaatatacgatgta | | | |
| | tttgaatgatgaagaagaaggcatacttgagatgaaacag | | | |
| | ttcttcaaaag [symbol] gggaaagcaacaaattccttatacgt | | | |
| | ttaattacaaatctgagttatttgatgtaagcaatccgtt | | | |
| | ttttagtaatgaaaccaaaattacatttgagaatgaagta | | | |
| | ttattaaccgcaaagcgtagttttttagatatttcaaaaa | | | |
| | gtaaactgactaaaaaacg [symbol] ggaaaaacacaatatac | | | |
| | acattcacagtactagagtagagaaagaaatattaatagc | | | |
| | catttacttacaatgcatgataaacaagcaaacacaataa | | | |
| | atgaagtatttaggtgtagtataaatgaatcaaaataataatt | | | |
| | gatttaaccattaacgaataaagattttagtacaaatata | | | |
| | ccctattatcataactgctaaaaaagatagtgaaggcaac | | | |
| | aaaacaaaccatattgacaccattttagctgt [symbol] gaaa | | | |
| | ctaaaagagaaatattggaagcaagccatagcagaatat | | | |
| | gaaaaacgtttaggcccatacaccaagatagacatcatag | | | |
| | aagttccagacgaaaaagcaccagaaaatatgagcgacaa | | | |
| | agaaattgagcaagtaaaa [symbol] ccaacgaat | | | |
| | actagccaaaatcaaaccacaatccacagtcattacatta | | | |
| | gaaataca [symbol] ggaaagatgctatcttccgaaggattggcc | | | |
| | caagag[ttgaaccaacgcatgaccca [symbol] gcaaagc | | | |
| | gactttgtattcgtcat [symbol] cgg]at | | | |
| | ----- | | | |
| | caccatttt[agctgt [symbol] gaaactaaaagagaaatattg | | | |
| | gaagcaagccatagcagaa]tatgaaaaacgtttaggccc | | | |
| | atacaccaagatagacatcatagaagttccagacgaaaaa | | | |
| | gca[ccagaaaatatgagcgacaaagaaattgagcaagta | | | |
| | [symbol] ccaacgaatactagccaaaatca | | | |
| | aaccacaatccacagtcattacattagaaatacaa[ggaa | | | |
| | agatgctatcttccgaaggattggcccaagag[ttgaaccaacgca | | | |
| | tgaccca [symbol] gcaaagcgactttgtattcgtcattgg]cggat | | | |
| SEQ ID | caccatttt[agctgt [symbol] gaaactaaaagagaaa[tatt | | | |
| NO: 27 | ggaagcaagccatagcagaa]tatgaaaaacgtttaggcc | | | |
| | ca[tacaccaagatagacatcatagaa]gttccagacgaa | | | |
| | aaagca[ccagaaaatatgagcgacaaagaa]attgagca | | | |
| | agtaaaa [symbol] ccaacgaatactagccaaaat | | | |
| | caaaccacaatccacagtcattacattagaaatacaagga | | | |
| | aagatgctatcttccgaaggattggcccaagagt[tgaac | | | |
| | caacgcatgacccaagggcaaagcgactttgtattcgtca | | | |
| | t [symbol] ]at | | | |
| SEQ ID | aatcaaaccacaatccaca[gtcattacattagaaataca | S. pyogenes | | gRNA_variant- |
| NO: 28 | agg][aaagatgctatcttccgaaggattgg][cccaag | | | SPm |
| | agttgaaccaacgcatgacccaaggg][caaagcgacttt | | | |
| | gtattcgtcat [symbol] [symbol] ggcgg]atTGTACAAAAAAGCAGG | | | |
| | CTTTAAAGGAACCAATTCAGTCGACTGGATCCGGTACCAA | | | |
| | GGTCGGGCAGGAAGAGGGCCTATTTCCCATGATTCCTTCA | | | |
| | TATTTGCATATACGATACAAGGCTGTTAGAGAGATAATTA | | | |
| | GAATTAATTTGACTGTAAACACAAAGATATTAGTACAAAA | | | |
| | TACGTGACGTAGAAAGTAATAATTTCTTGGGTAGTTTGCA | | | |
| | GTTTTAAAATTATGTTTTAAAATGGACTATCATATGCTTA | | | |
| | CCGTAACTTGAAAGTATTTCGATTTCTTGGCTTTATATAT | | | |
| | CTTGTGGAAAGGACGAAACACCGNNNNNNNNNNNNNNNNN | | | |
| | NNNGTTTTAGAGCTAGAAATAGCAAGTTAAAATAAGGCTA | | | |
| | GTCCGTTATCAACTTGAAAAAGTGGCACCGAGTCGGTGCT | | | |
| | TTTTTTT | | | |
| SEQ ID | TGTACAAAAAAGCAGGCTTTAAAGGAACCAATTCAGTCGA | S. thermophilus | | gRNA_variant- |
| NO: 29 | CTGGATCCGGTACCAAGGTCGGGCAGGAAGAGGGCCTATT | | | ST1f1 |
| | TCCCATGATTCCTTCATATTTGCATATACGATACAAGGCT | | | |
| | GTTAGAGAGATAATTAGAATTAATTTGACTGTAAACACAA | | | |
| | AGATATTAGTACAAAATACGTGACGTAGAAAGTAATAATT | | | |
| | TCTTGGGTAGTTTGCAGTTTTAAAATTATGTTTTAAAATG | | | |
| | GACTATCATATGCTTACCGTAACTTGAAAGTATTTCGATT | | | |
| | TCTTGGCTTTATATATCTTGTGGAAAGGACGAAACACCGN | | | |
| | NNNNNNNNNNNNNNNNNNNGTTTTTGTACTCTCAAGATTT | | | |
| | AAGTAACTGTACAACGAAACTTACACAGTTACTTAAATCT | | | |
| | TGCAGAAGCTACAAAGATAAGGCTTCATGCCGAAATCAAC | | | |
| | ACCCTGTCATTTTATGGCAGGGTGTTTTTTT | | | |
| SEQ ID | TGTACAAAAAAGCAGGCTTTAAAGGAACCAATTCAGTCGA | S. thermophilus | | gRNA_variant- |
| NO: 30 | CTGGATCCGGTACCAAGGTCGGGCAGGAAGAGGGCCTATT | | | ST1m1 |
| | TCCCATGATTCCTTCATATTTGCATATACGATACAAGGCT | | | |
| | GTTAGAGAGATAATTAGAATTAATTTGACTGTAAACACAA | | | |
| | AGATATTAGTACAAAATACGTGACGTAGAAAGTAATAATT | | | |
| | TCTTGGGTAGTTTGCAGTTTTAAAATTATGTTTTAAAATG | | | |
| | GACTATCATATGCTTACCGTAACTTGAAAGTATTTCGATT | | | |
| | TCTTGGCTTTATATATCTTGTGGAAAGGACGAAACACCGN | | | |
| | NNNNNNNNNNNNNNNNNNNGTTTTTGTACTCTCAGAAATG | | | |
| | CAGAAGCTACAAAGATAAGGCTTCATGCCGAAATCAACAC | | | |
| | CCTGTCATTTTATGGCAGGGTGTTTTTTT | | | |
| SEQ ID | TGTACAAAAAAGCAGGCTTTAAAGGAACCAATTCAGTCGA | N. meningitidis | | gRNA_variant- |
| NO: 31 | CTGGATCCGGTACCAAGGTCGGGCAGGAAGAGGGCCTATT | | | NMf |
| | TCCCATGATTCCTTCATATTTGCATATACGATACAAGGCT | | | |
| | GTTAGAGAGATAATTAGAATTAATTTGACTGTAAACACAA | | | |
| | AGATATTAGTACAAAATACGTGACGTAGAAAGTAATAATT | | | |
| | TCTTGGGTAGTTTGCAGTTTTAAAATTATGTTTTAAAATG | | | |
| | GACTATCATATGCTTACCGTAACTTGAAAGTATTTCGATT | | | |
| | TCTTGGCTTTATATATCTTGTGGAAAGGACGAAACACCGN | | | |
| | NNNNNNNNNNNNNNNNNNNGTTGTAGCTCCCTTTCTCATT | | | |
| | TCGCAGTGCTACAATGAAAATTGTCGCACTGCGAAATGAG | | | |
| | AACCGTTGCTACAATAAGGCCGTCTGAAAAGATGTGCCGC | | | |
| | AACGCTCTGCCCCTTAAAGCTTCTGCTTTAAGGGGCTTTT | | | |
| | TTT | | | |
| SEQ ID | TGTACAAAAAAGCAGGCTTTAAAGGAACCAATTCAGTCGA | N. meningitidis | | gRNA_variant- |
| NO: 32 | CTGGATCCGGTACCAAGGTCGGGCAGGAAGAGGGCCTATT | | | NMm1 |
| | TCCCATGATTCCTTCATATTTGCATATACGATACAAGGCT | | | |
| | GTTAGAGAGATAATTAGAATTAATTTGACTGTAAACACAA | | | |
| | AGATATTAGTACAAAATACGTGACGTAGAAAGTAATAATT | | | |
| | TCTTGGGTAGTTTGCAGTTTTAAAATTATGTTTTAAAATG | | | |
| | GACTATCATATGCTTACCGTAACTTGAAAGTATTTCGATT | | | |
| | TCTTGGCTTTATATATCTTGTGGAAAGGACGAAACACCGN | | | |
| | NNNNNNNNNNNNNNNNNNNGTTGTAGCTCCCTTTCTCGAA | | | |
| | AGAGAACCGTTGCTACAATAAGGCCGTCTGAAAAGATGTG | | | |
| | CCGCAACGCTCTGCCCCTTAAAGCTTCTGCTTTAACGGGC | | | |
| | TTTTTTT | | | |

### Reporter Molecules for Detecting High-Specificity Binding to a Target Nucleic Acid Sequence

The present disclosure provides fusion proteins, in particular fusion protein pairs, wherein each fusion protein pair includes a first fusion protein comprising a first target sequence specific binding protein and a first half of a split-reporter molecule, such as a split-reporter protein and includes a second fusion protein comprising a second target sequence specific binding protein and a second half of a split-reporter molecule, such as a split-reporter protein. When both fusion proteins of a fusion protein pair bind to the corresponding target sequences within a target nucleic acid, the two halves of the split-reporter molecule are brought into juxtaposition thereby regenerating a functional reporter molecule. Thus, the target specific binding of a pair of fusion proteins to a target sequence can be determined by detecting a signal that is generated by the regenerated reporter molecule.

Exemplified herein are split-reporter molecules such as a split-fluorescent reporter molecules, split-luminescent reporter molecules, Förster resonance energy transfer (FRET) reporter molecules, and Bioluminescence Resonance Energy Transfer (BRET) reporter molecules.

Split-protein systems are described, generally, in Shekhawat and Ghosh, Curr Opin Chem Biol 15(6):789-797 (2011). Various suitable split-reporter protein systems than may be adapted for use in the fusion proteins described herein are presented in Lee et al., PLOS One, 7(8):e43820 (2012) (split-intein); Kato and Jones, Methods in Mol Biol 655:357-376 (2010) (split-luciferase complementation assay); Kaddoum et al., BioTechniques 49:727-736 (2010) (split-green fluorescent protein (GFP) staining for protein detection and localization in mammalian cells); Fujikawa and Kato, Plant J 52(1):185-95 (2007) (split-luciferase complementation assay); Cabantous et al., Scientific Reports 3(2854):1 (2013) (a protein-protein interaction sensor based on split-GFP association); Kent et al., JACS 130:9664-96656 (2008) (deconstructing GFP); Kent et al., JACS 131:15988-15989 (2009) (synthetic control of GFP); Paulmurugan and Gambhir, Canc Res 65:7413-7420 (2005) (fusion proteins with split-Renilla luciferase and with split-enhanced green fluorescent protein (split-EGFP); and Wang et al., J Biol Chem 275:18418-23 (2000) (split-transducin-like enhancer (TLE)).

In addition to these split-protein and split-reporter protein systems, other split-proteins are generally known and are readily available in the art including, for example, split-dihydrofolate reductase (DHJFR), split-beta-lactamase, split-Ga14 (yeast two-hybrid system), split-tobacco etch virus protease (TEV), split-ubiquitin, and split-beta-galactosidase (LacZ).

Provided herein are fusion protein pairs wherein a first reporter molecule comprises the C-terminus of split-Renilla reniformis luciferase and wherein a second reporter molecule comprises the N-terminus of split-Renilla reniformis luciferase. It will be understood that when the C-terminus of split-Renilla reniformis luciferase is brought into juxtaposition of the N-terminus of split-Renilla reniformis luciferase, the resulting reformed luciferase can interact its substrate coelenterazine to produce light having a peak emission wavelength of 482 nm.

Also provided herein are fusion protein pairs wherein a first reporter molecule comprises the N-terminus of split-enhanced green fluorescent protein (EGFP) and wherein a second reporter molecule comprises the C-terminus of split-enhanced GFP. It will be understood that when the N-terminus of split-EGFP is brought into juxtaposition of the C-terminus of split-EGFP, the resulting reformed enhanced GFP produce light having a peak emission wavelength of 395 nm and 475 nm when exposed to light in the blue to ultraviolet range. See, Prendergast and Mann, Biochemistry 17(17):3448-53 (1978) and Tsien, Annu Rev Biochem 67:509-44 (1998).

Also provided herein are fusion protein pairs wherein a first reporter molecule comprises a cyan fluorescent protein (CFP) and wherein a second reporter molecule comprises a yellow fluorescent protein (YFP). It will be understood that when the CFP is brought into juxtaposition of the YFP by the binding of a first fusion protein comprising a CFP reporter molecule to a first region of a target DNA sequence and the binding of a second fusion protein comprising a YFP reporter molecule to a second region of a target DNA sequence, the 480 nm fluorescent signal emitted from CFP following exposure to light of 440 nm can excite the YFP to emit light of 535 nm via Förster resonance energy transfer (FRET), the detection of which the close association of CFP and YFP and, hence, the binding of both the first and second fusion proteins to the target DNA sequence.

In an alternative embodiment of the present disclosure, rather than employing a split-fluorescent protein as a reporter molecule, distinct fluorophores can be fused to a target specific nucleic acid binding protein to generate fusion proteins exhibiting different fluorescent characteristics. Thus, if each member of a fusion protein pair employs a distinct fluorophore (in contrast to a split-fluorophore protein) the binding of each fusion protein to a target nucleic acid will bring the two distinct fluorophores into proximity spatially. If the fluorophores are oriented in a manner that exposes the fluorophores to one another, which is ensured by the design of each fluorophore-target specific protein, then the energy transfer from the excited donor fluorophore will result in a change in the fluorescent intensities or lifetimes of the fluorophores.

As used herein, the terms “Förster resonance energy transfer,” “Fluorescence resonance energy transfer,” and “FRET” refer to the energy transfer between two fluorophores (i.e., an excited (donor) fluorophore to a nearby acceptor). A donor fluorophore, initially in its electronic excited state, may transfer energy to an acceptor fluorophore through nonradiative dipoledipole coupling. The efficiency of this energy transfer is inversely proportional to the sixth power of the distance between donor and acceptor making FRET extremely sensitive to small distances. Measurements of FRET efficiency can be used to determine if two fluorophores are within a certain distance of each other.

### Fusion Proteins Comprising a Nucleic Acid Binding Protein and a Split-Reporter Molecules for Detecting a DNA Sequence in a Sample

The compositions, systems, and methods described herein employ one or more fusion protein(s), each of which comprises a DNA sequence-specific binding protein and a reporter molecule, wherein the binding protein is operably linked to the reporter molecule.

Exemplified herein are fusion proteins comprising a sequence-specific nucleic acid binding proteins, such as sequence-specific three prime repair exonucleases (“TREX”), sequence specific Cas9 proteins (e.g., CRISPRs), sequence specific transcription activator-like enhancer (“TALE”) proteins, sequence specific homing endonucleases (“HE”; a/k/a meganucleases), and sequence specific zinc finger (“ZF”) proteins, which are operably linked to one half of a split-reporter molecule, such as a split-fluorescent reporter molecule, a split-luminescent reporter molecule, a Förster resonance energy transfer (FRET) reporter molecule, or a Bioluminescence Resonance Energy Transfer (BRET) reporter molecule.

Fusion proteins, or DNA binding portions thereof, having suitable target DNA sequence-specificity may be identified by a yeast surface display strategy, combined with high-throughput cell sorting for desirable DNA cleavage specificity. A series of protein-DNA ‘modules’, which correspond to sequential pockets of contacts that extend across the entire target site, may be systematically randomized in separate libraries. Each library may then be systematically sorted for populations of enzymes that can specifically cleave each possible DNA variant within each module, and each sorted population deep-sequenced and archived for subsequent enzyme assembly and design.

Within these embodiments, each TAL effector binding protein specifically targets a DNA sequence, thereby bringing a reporter molecule of a first fusion protein in juxtaposition with a second fusion protein on adjacent fluorescent or luminescent technology in contact which each other, allowing the production of light. This production of light is due to regained activity of the luminescent or fluorescent report, allowing it to catalyze its corresponding substrate and give off light as a by-product, or by excited by a laser, and by FRET or BRET technology allowing for the production of excited photons.

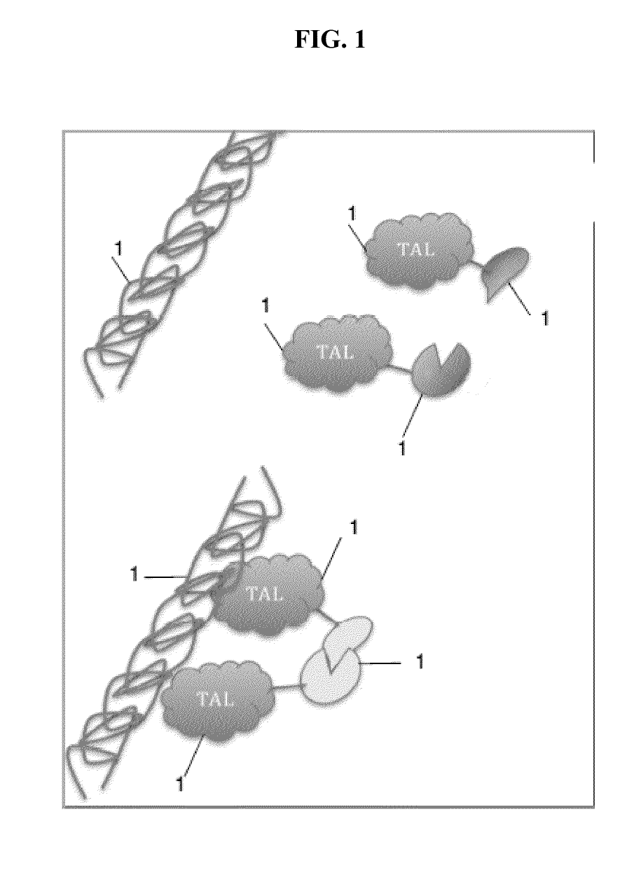

One embodiment of this disclosure (see FIG. 1) permits the detection of a target nucleic acid by employing a fusion protein pair comprising a first fusion protein that contains the N-terminus of split-Renilla reniformis luciferase, which is linked to a first TAL effector that targets a first target nucleotide sequence and a second fusion protein that contains the C-terminus of split-Renilla reniformis luciferase, which is linked to a second TAL effector that targets a second target nucleotide sequence. When the first and second fusion proteins are contacted with a target nucleic acid having a first target nucleotide sequence that is adjacent to a second target nucleotide sequence, the N-terminus and C-terminus of the split-Renilla reniformis luciferase are brought into juxtaposition such that a functional Renilla reniformis luciferase is reformed. Thus, the presence of a target nucleic acid can be determined by detecting the generation of a fluorescent signal in the presence of coelenterazine.

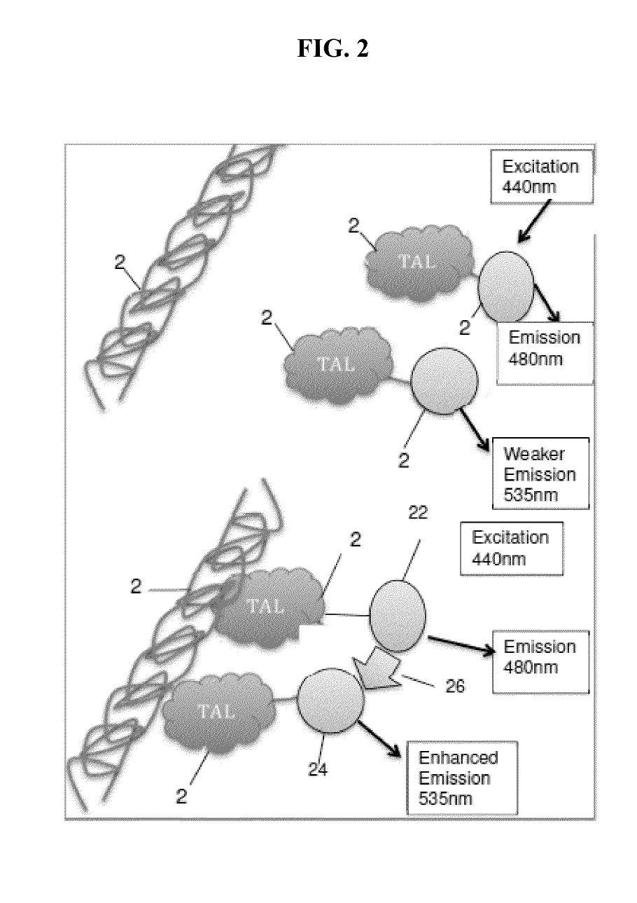

Another embodiment of this disclosure (see FIG. 2) permits the detection of a target nucleic acid with a fusion protein pair wherein a first fusion protein comprises a first half of a split-cyan fluorescent protein that is linked to a first TAL effector having target specificity for one nucleotide sequence within the target nucleic acid and a second fusion protein comprises a second half of a cyan fluorescent protein that is linked to a second TAL effector having target specificity for an adjacent nucleotide sequence within the target nucleic acid. When the first and second fusion proteins are contacted in the presence of calcium ions with the target nucleic acid, the first and second halves of the split-cyan fluorescent protein are brought into juxtaposition such that a function cyan fluorescent protein is formed that, when exposed to an external light beam, a high level of photon excitation can be detected, which photon excitation corresponds directly with to the presence of the target nucleic acid. This embodiment can also substitute a photon producing chromophore, like a variant Renilla reniformis luciferase, instead of cyan fluorescent protein obliterating the need for outside light excitation.

A further embodiment of this disclosure permits the detection of a target nucleic acid with a fusion protein pair wherein a first fusion protein comprises a first half of a split-enhanced green fluorescent protein (EGFP), which is encoded by the nucleotide sequence of SEQ ID NO: 4, which first half of split-EGFP is linked to a Cas9 protein, which is encoded by the nucleotide sequence of SEQ ID NO: 2 (SpyCas9) and having a tracrRNA having target specificity for the nucleotide sequence of SEQ ID NO: 7 and wherein a second fusion protein comprises a second half of a split-EGFP, which is encoded by the nucleotide sequence of SEQ ID NO: 5, which second half of split-EGFP is linked to the Cas9 protein, which is encoded by the nucleotide sequence of SEQ ID NO: 2 (SpyCas9) and having a tracrRNA having target specificity for the nucleotide sequence of SEQ ID NO: 8. See, Table 2. When the first and second fusion proteins are contacted with a target nucleic acid having a target nucleotide sequence of SEQ ID NO: 7 that is adjacent to the target nucleotide sequence of SEQ ID NO: 8, the first and second halves of the split-EGFP are brought into juxtaposition such that a functional EGFP protein is reformed. Thus, when exposed to an external light beam, a high level of photon excitation can be detected, which photon excitation corresponds directly with to the presence of the target nucleic acid. This embodiment can also substitute a photon producing chromophore, like a variant Renilla reniformis luciferase, instead of enhanced green fluorescent protein.

The exemplary fusion construct presented in Table 2 can be used to target the mecA gene in Methicillin-resistant Staphylococcus aureus to distinguish it from other strains of Staphylococcus aureus.

It will be understood that these embodiments are provided by way of example, not limitation, and that a wide variety of fusion protein pairs are contemplated wherein a fusion protein pair includes a first fusion protein and a second fusion protein, wherein the first fusion protein comprises a first target sequence specific nucleic acid binding protein linked to a first half of a split-reporter molecule, such as a reporter protein and wherein the second fusion protein comprises a second target sequence specific nucleic acid binding protein linked to a second half of a split-reporter molecule, such as a reporter protein.

The present disclosure contemplates the use of a wide variety of split-reporter molecules, in particular split-reporter proteins, such as a split-luminescent reporter protein or a split-fluorescent reporter protein, and a wide variety of target sequence specific nucleic acid binding proteins, such as sequence-specific (“TREX”) proteins, sequence specific Cas9 proteins (e.g., CRISPRs), sequence specific transcription activator-like enhancer (“TALE”) proteins, sequence specific homing endonucleases (“HE”; a/k/a meganucleases), and sequence specific zinc finger (“ZF”) proteins.

The present disclosure further contemplates that alternative reporter proteins may be prepared as split-reporter proteins by following the guidance presented herein and as otherwise available to those of skill in the art. Considerations for the design of split-reporter proteins for use in the presently-disclosed fusion proteins include: (1) ensuring that the first and second halves of a reporter protein are able to associate with one another to reform a functional protein when each half is linked to a target sequence specific nucleic acid binding protein (structural information and the location of interaction surfaces may be considered) and (2) the first and second halves of a reporter protein must not significantly alter the folding, production, localization, stability and/or biological function (i.e., nucleic acid binding specificity/affinity) of the target sequence specific nucleic acid binding protein to which it is linked as compared to a corresponding wild-type protein.

It will be understood that the selection of fluorescent split-reporter protein requires consideration for the cellular environment in which the fusion protein is expressed. For example, GFP can be used in E. coli cells, while YFP is suitable for use in mammalian cells. Kerppola, Nat Methods 3:969-971 (2006).

Yellow fluorescent protein (YFP) can serve as a split-reporter protein and is typically separated into an N-terminal half having amino acids 1-154 and a C-terminal half having amino acids 155-238. These fragments of YFP are highly efficient in complementation when fused to many proteins, including target specific nucleic acid binding proteins. Moreover they produce low levels of fluorescence when fused to non-interacting proteins.

It is generally advisable to generate alternative combinations of first and second target nucleic acid specific proteins and first and second halves of split-reporter proteins. Thus, each target protein can be fused to both the N- and C-terminal fragments of the split-reporter protein in turn, and the fragments can be fused at each of the N- and C-terminal ends of the target proteins. This results in a total of eight permutations per fusion protein, with interactions being tested as follows:
(1) N-terminal fragment fused at the N-terminal protein 1+C-terminal fragment fused at the N-terminal protein 2
(2) N-terminal fragment fused at the N-terminal protein 1+C-terminal fragment fused at the C-terminal protein 2
(3) N-terminal fragment fused at the C-terminal protein 1+C-terminal fragment fused at the N-terminal protein 2
(4) N-terminal fragment fused at the C-terminal protein 1+C-terminal fragment fused at the C-terminal protein 2
(5) C-terminal fragment fused at the N-terminal protein 1+N-terminal fragment fused at the N-terminal protein 2
(6) C-terminal fragment fused at the N-terminal protein 1+N-terminal fragment fused at the C-terminal protein 2
(7) C-terminal fragment fused at the C-terminal protein 1+N-terminal fragment fused at the N-terminal protein 2
(8) C-terminal fragment fused at the C-terminal protein 1+N-terminal fragment fused at the C-terminal protein 2

Fusion proteins of the present disclosure may employ one or more linkers, such as a linker peptide, to separate the target sequence specific nucleic acid binding protein from the first or second half (e.g., N- or C-terminal portion) of a split-reporter protein. Such a linker can, for example, reduce steric hindrances between those fusion protein components. When designing a linker sequence, it is important to consider the solubility, length, and amino acid composition of the linker to ensure that the split-reporter protein halves exhibit sufficient flexibility and freedom of movement so that the first and second split-reporter protein halves can come into juxtaposition and reform a functional reporter protein.

Exemplified herein are short (i.e. four to 75 amino acids) linkers comprising from about one peptide having the sequence GGGG or GGGGX to about 15 consecutive peptides having the sequence GGGG or GGGGX, wherein X is independently selected from A, V, G, L, I, P, Y and S. Exemplary suitable linkers include the four amino acid flexible linker GGGG, the five amino acid flexible linker GGGGS, the 15 amino acid flexible linkers GGGGGGGGGGGGGGG, GGGGSGGGGSGGGGS, and GGGGSGGGGSGGGGT, the 19 amino acid linker LGGGGSGGGGSGGGGSAAA, and the 25 amino acid linker LSGGGGSGGGGSGGGGSGGGGSAAA.

Other linkers that may be satisfactorily employed with the fusion proteins disclosed herein include linkers comprising the sequences LAAA, RSIAT, RPACKIPNDLKQKVMNH, AAANSSIDLISVPVDSR, and LQGGSGGGGSGGGGY, which have been used successfully in various bimolecular fluorescence applications.

Still further linkers that may be satisfactorily employed with the fusion proteins disclosed herein include the helix-forming peptide linkers having the amino acid sequence A(EAAAK)ₙA (n=−25), such as AEAAAKEAAAKEAAAKA, LAEAAAKEAAAKAAA, LAEAAAKEAAAKEAAAKAAA, LAEAAAKEAAAKEAAAKEAAAKAAA, LAEAAAKEAAAKEAAAKEAAAKEAAAKAAA, LFNKEQQNAFYEILHLPNLNEEQRNGFIQSLKDDPSQSANLLAEAKKLNDAQAAA, which linkers control the distance and reduce the interference between constituent green fluorescent protein variant EBFP and EGFP subunits. See, Arai et al., Protein Engineering 14(8):529-532 (2001).

TABLE 2
Sequence Elements for an Exemplary Targeting Protein split-Reporter Protein Construct

| Sequence | Sequence | |
|---|---|---|
| Identifier | Description | Nucleotide Sequence (5′-3′) |
| SEQ ID | Promoter | TTCTAGAGCACAGCTAACACCACGTCGTCCCTATCTGCTGCCCTAGGTCTATGAGTGGTTGCTGGATAACTTTA |
| NO: 1 | | CGGGCATGCATAAGGCTCGTATGATATATTCAGGGAGACCACAACGGTTTCCCTCTACAAATAATTTTGTTTAA |
| | | CTTTTACTAGAG |
| SEQ ID | SpyCas9 | ATGGACAAGAAGTACTCCATTGGGCTCGCTATCGGCACAAACAGCGTCGGCTGGGCCGTCATTACGGACGAGTA |
| NO: 2 | | CAAGGTGCCGAGCAAAAAATTCAAAGTTCTGGGCAATACCGATCGCCACAGCATAAAGAAGAACCTCATTGGCG |
| | | CCCTCCTGTTCGACTCCGGGGAGACGGCCGAAGCCACGCGGCTCAAAAGAACAGCACGGCGCAGATATACCCGC |
| | | AGAAAGAATCGGATCTGCTACCTGCAGGAGATCTTTAGTAATGAGATGGCTAAGGTGGATGACTCTTTCTTCCA |
| | | TAGGCTGGAGGAGTCCTTTTTGGTGGAGGAGGATAAAAAGCACGAGCGCCACCCAATCTTTGGCAATATCGTGG |
| | | ACGAGGTGGCGTACCATGAAAAGTACCCAACCATATATCATCTGAGGAAGAAGCTTGTAGACAGTACTGATAAG |
| | | GCTGACTTGCGGTTGATCTATCTCGCGCTGGCGCATATGATCAAATTTCGGGGACACTTCCTCATCGAGGGGGA |
| | | CCTGAACCCAGACAACAGCGATGTCGACAAACTCTTTATCCAACTGGTTCAGACTTACAATCAGCTTTTCGAAG |
| | | AGAACCCGATCAACGCATCCGGAGTTGACGCCAAAGCAATCCTGAGCGCTAGGCTGTCCAAATCCCGGCGGCTC |
| | | GAAAACCTCATCGCACAGCTCCCTGGGGAGAAGAAGAACGGCCTGTTTGGTAATCTTATCGCCCTGTCACTCGG |
| | | GCTGACCCCCAACTTTAAATCTAACTTCGACCTGGCCGAAGATGCCAAGCTTCAACTGAGCAAAGACACCTACG |
| | | ATGATGATCTCGACAATCTGCTGGCCCAGATCGGCGACCAGTACGCAGACCTTTTTTTGGCGGCAAAGAACCTG |
| | | TCAGACGCCATTCTGCTGAGTGATATTCTGCGAGTGAACACGGAGATCACCAAAGCTCCGCTGAGCGCTAGTAT |
| | | GATCAAGCGCTATGATGAGCACCACCAAGACTTGACTTTGCTGAAGGCCCTTGTCAGACAGCAACTGCCTGAGA |
| | | AGTACAAGGAAATTTTCTTCGATCAGTCTAAAAATGGCTACGCCGGATACATTGACGGCGGAGCAAGCCAGGAG |
| | | GAATTTTACAAATTTATTAAGCCCATCTTGGAAAAAATGGACGGCACCGAGGAGCTGCTGGTAAAGCTTAACAG |
| | | AGAAGATCTGTTGCGCAAACAGCGCACTTTCGACAATGGAAGCATCCCCCACCAGATTCACCTGGGCGAACTGC |
| | | ACGCTATCCTCAGGCGGCAAGAGGATTTCTACCCCTTTTTGAAAGATAACAGGGAAAAGATTGAGAAAATCCTC |
| | | ACATTTCGGATACCCTACTATGTAGGCCCCCTCGCCCGGGGAAATTCCAGATTCGCGTGGATGACTCGCAAATC |
| | | AGAAGAGACCATCACTCCCTGGAACTTCGAGGAAGTCGTGGATAAGGGGGCCTCTGCCCAGTCCTTCATCGAAA |
| | | GGATGACTAACTTTGATAAAAATCTGCCTAACGAAAAGGTGCTTCCTAAACACTCTCTGCTGTACGAGTACTTC |
| | | ACAGTTTATAACGAGCTCACCAAGGTCAAATACGTCACAGAAGGGATGAGAAAGCCAGCATTCCTGTCTGGAGA |
| | | GCAGAAGAAAGCTATCGTGGACCTCCTCTTCAAGACGAACCGGAAAGTTACCGTGAAACAGCTCAAAGAAGACT |
| | | ATTTCAAAAAGATTGAATGTTTCGACTCTGTTGAAATCAGCGGAGTGGAGGATCGCTTCAACGCATCCCTGGGA |
| | | ACGTATCACGATCTCCTGAAAATCATTAAAGACAAGGACTTCCTGGACAATGAGGAGAACGAGGACATTCTTGA |
| | | GGACATTGTCCTCACCCTTACGTTGTTTGAAGATAGGGAGATGATTGAAGAACGCTTGAAAACTTACGCTCATC |
| | | TCTTCGACGACAAAGTCATGAAACAGCTCAAGAGGCGCCGATATACAGGATGGGGGCGGCTGTCAAGAAAACTG |
| | | ATCAATGGGATCCGAGACAAGCAGAGTGGAAAGACAATCCTGGATTTTCTTAAGTCCGATGGATTTGCCAACCG |
| | | GAACTTCATGCAGTTGATCCATGATGACTCTCTCACCTTTAAGGAGGACATCCAGAAAGCACAAGTTTCTGGCC |
| | | AGGGGGACAGTCTTCACGAGCACATCGCTAATCTTGCAGGTAGCCCAGCTATCAAAAAGGGAATACTGCAGACC |
| | | GTTAAGGTCGTGGATGAACTCGTCAAAGTAATGGGAAGGCATAAGCCCGAGAATATCGTTATCGAGATGGCCCG |
| | | AGAGAACCAAACTACCCAGAAGGGACAGAAGAACAGTAGGGAAAGGATGAAGAGGATTGAAGAGGGTATAAAAG |
| | | AACTGGGGTCCCAAATCCTTAAGGAACACCCAGTTGAAAACACCCAGCTTCAGAATGAGAAGCTCTACCTGTAC |
| | | TACCTGCAGAACGGCAGGGACATGTACGTGGATCAGGAACTGGACATCAATCGGCTCTCCGACTACGACGTGGC |
| | | TGCTATCGTGCCCCAGTCTTTTCTCAAAGATGATTCTATTGATAATAAAGTGTTGACAAGATCCGATAAAGCTA |
| | | GAGGGAAGAGTGATAACGTCCCCTCAGAAGAAGTTGTCAAGAAAATGAAAAATTATTGGCGGCAGCTGCTGAAC |
| | | GCCAAACTGATCACACAACGGAAGTTCGATAATCTGACTAAGGCTGAACGAGGTGGCCTGTCTGAGTTGGATAA |
| | | AGCCGGCTTCATCAAAAGGCAGCTTGTTGAGACACGCCAGATCACCAAGCACGTGGCCCAAATTCTCGATTCAC |
| | | GCATGAACACCAAGTACGATGAAAATGACAAACTGATTCGAGAGGTGAAAGTTATTACTCTGAAGTCTAAGCTG |
| | | GTCTCAGATTTCAGAAAGGACTTTCAGTTTTATAAGGTGAGAGAGATCAACAATTACCACCATGCGCATGATGC |
| | | CTACCTGAATGCAGTGGTAGGCACTGCACTTATCAAAAAATATCCCAAGCTTGAATCTGAATTTGTTTACGGAG |
| | | ACTATAAAGTGTACGATGTTAGGAAAATGATCGCAAAGTCTGAGCAGGAAATAGGCAAGGCCACCGCTAAGTAC |
| | | TTCTTTTACAGCAATATTATGAATTTTTTCAAGACCGAGATTACACTGGCCAATGGAGAGATTCGGAAGCGACC |
| | | ACTTATCGAAACAAACGGAGAAACAGGAGAAATCGTGTGGGACAAGGGTAGGGATTTCGCGACAGTCCGGAAGG |
| | | TCCTGTCCATGCCGCAGGTGAACATCGTTAAAAAGACCGAAGTACAGACCGGAGGCTTCTCCAAGGAAAGTATC |
| | | CTCCCGAAAAGGAACAGCGACAAGCTGATCGCACGCAAAAAAGATTGGGACCCCAAGAAATACGGCGGATTCGA |
| | | TTCTCCTACAGTCGCTTACAGTGTACTGGTTGTGGCCAAAGTGGAGAAAGGGAAGTCTAAAAAACTCAAAAGCG |
| | | TCAAGGAACTGCTGGGCATCACAATCATGGAGCGATCAAGCTTCGAAAAAAACCCCATCGACTTTCTCGAGGCG |
| | | AAAGGATATAAAGAGGTCAAAAAAGACCTCATCATTAAGCTTCCCAAGTACTCTCTCTTTGAGCTTGAAAACGG |
| | | CCGGAAACGAATGCTCGCTAGTGCGGGCGAGCTGCAGAAAGGTAACGAGCTGGCACTGCCCTCTAAATACGTTA |
| | | ATTTCTTGTATCTGGCCAGCCACTATGAAAAGCTCAAAGGGTCTCCCGAAGATAATGAGCAGAAGCAGCTGTTC |
| | | GTGGAACAACACAAACACTACCTTGATGAGATCATCGAGCAAATAAGCGAATTCTCCAAAAGAGTGATCCTCGC |
| | | CGACGCTAACCTCGATAAGGTGCTTTCTGCTTACAATAAGCACAGGGATAAGCCCATCAGGGAGCAGGCAGAAA |
| | | ACATTATCCACTTGTTTACTCTGACCAACTTGGGCGCGCCTGCAGCCTTCAAGTACTTCGACACCACCATAGAC |
| | | AGAAAGCGGTACACCTCTACAAAGGAGGTCCTGGACGCCACACTGATTCATCAGTCAATTACGGGGCTCTATGA |
| | | AACAAGAATCGACCTCTCTCAGCTCGGTGGAGACTAA |
| SEQ ID | Linker | GGUGGUGGAGGA |
| NO: 3 | | |
| SEQ ID | C-terminus | AAGAACGGCATCAAGGTGAACTTCAAGATCCGCCACAACATCGAGGACGGCAGCGTGCAGCTCGCCGACCACTA |
| NO: 4 | Fragment of | CCAGCAGAACACCCCCATCGGCGACGGCCCCGTGCTGCTGCCCGACAACCACTACCTGAGCACCCAGTCCGCCC |
| | Split-EGFP | TGAGCAAAGACCCCAACGAGAAGCGCGATCACATGGTCCTGCTGGAGTTCGTGACCGCCGCCGGGATCACTCTC |
| | | GGCATGGACGAGCTGTACAAG |
| SEQ ID | N-terminal | ATGGTGAGCAAGGGCGAGGAGCTGTTCACCGGGGTGGTGCCCATCCTGGTCGAGCTGGACGGCGACGTAAACGG |
| NO: 5 | Fragment of | CCACAAGTTCAGCGTGTCCGGCGAGGGCGAGGGCGATGCCACCTACGGCAAGCTGACCCTGAAGTTCATCTGCA |
| | Split-EGFP | CCACCGGCAAGCTGCCCGTGCCCTGGCCCACCCTCGTGACCACCCTGACCTACGGCGTGCAGTGCTTCAGCCGC |
| | | TACCCCGACCACATGAAGCAGCACGACTTCTTCAAGTCCGCCATGCCCGAAGGCTACGTCCAGGAGCGCACCAT |
| | | CTTCTTCAAGGACGACGGCAACTACAAGACCCGCGCCGAGGTGAAGTTCGAGGGCGACACCCTGGTGAACCGCA |
| | | TCGAGCTGAAGGGCATCGACTTCAAGGAGGACGGCAACATCCTGGGGCACAAGCTGGAGTACAACTACAACAGC |
| | | CACAACGTCTATATCATGGCCGACAAGCAG |
| SEQ ID | TracrRNA | CTGATAAATTTCTTTGAATTTCTCCTTGATTATTTGTTATAAATGTTATAAAAT |
| NO: 6 | Promoter | |
| SEQ ID | C-phusion | TGAACCAACGCATGACCCAA |
| NO: 7 | Target | |
| | Sequence | |
| SEQ ID | N-phusion | GGAAAGATGCTATCTTCCGA |
| NO: 8 | Target | |
| | Sequence | |
| SEQ ID | TracrRNA | GTTGGAACCATTCAAAACAGCATAGCAAGTTAAAATAAGGCTAGTCCGTTATCAACTTGAAAAAGTGGCACCGA |
| NO: 9 | Precursor | GTCGGTGCTTTTTTT (Bold = TracrRNA Terminator) |
| SEQ ID | Terminator | TAAAAATGATAAAACAAGCGTTTTGAAAGCGCTTGTTTTTTT |
| NO: 10 | | |
| SEQ ID | J23100 | GACAATGAAAACGTTAGTCATGGCGCGCCTTGACGGCTAGCTCAGTCCTAGGTACAGTGCTAGCTTAAT |
| NO: 11 | Promoter | |
| SEQ ID | Origin of | GATCAAAGGATCTTCTTGAGATCCTTTTTTTCTGCGCGTAATCTTTTGCCCTGTAAACGAAAAAACCACCTGGG |
| NO: 12 | Replication | GAGGTGGTTTGATCGAAGGTTAAGTCAGTTGGGGAACTGCTTAACCGTGGTAACTGGCTTTCGCAGAGCACAGC |
| | | AACCAAATCTGTCCTTCCAGTGTAGCCGGACTTTGGCGCACACTTCAAGAGCAACCGCGTGTTTAGCTAAACAA |
| | | ATCCTCTGCGAACTCCCAGTTACCAATGGCTGCTGCCAGTGGCGTTTTACCGTGCTTTTCCGGGTTGGACTCAA |
| | | GTGAACAGTTACCGGATAAGGCGCAGCAGTCGGGCTGAACGGGGAGTTCTTGCTTACAGCCCAGCTTGGAGCGA |
| | | ACGACCTACACCGAGCCGAGATACCAGTGTGTGAGCTATGAGAAAGCGCCACACTTCCCGTAAGGGAGAAAGGC |
| | | GGAACAGGTATCCGGTAAACGGCAGGGTCGGAACAGGAGAGCGCAAGAGGGAGCGACCCGCCGGAAACGGTGGG |
| | | GATCTTTAAGTCCTGTCGGGTTTCGCCCGTACTGTCAGATTCATGGTTGAGCCTCACGGCTCCCACAGATGCAC |
| | | CGGAAAAGCGTCTGTTTATGTGAACTCTGGCAGGAGGGCGGAGCCTATGGAAAAACGCCACCGGCGCGGCCCTG |
| | | CTGTTTTGCCTCACATGTTAGTCCCCTGCTTATCCACGGAATCTGTGGGTAACTTTGTATGTGTCCGCAGCGC |
| SEQ ID | Antibiotic | ATGAGGGAAGCGGTGATCGCCGAAGTATCGACTCAACTATCAGAGGTAGTTGGCGTCATCGAGCGCCATCTCGA |
| NO: 13 | Resistance | ACCGACGTTGCTGGCCGTACATTTGTACGGCTCCGCAGTGGATGGCGGCCTGAAGCCACACAGTGATATTGATT |
| | | TGCTGGTTACGGTGACCGTAAGGCTTGATGAAACAACGCGGCGAGCTTTGATCAACGACCTTTTGGAAACTTCG |
| | | GCTTCCCCTGGAGAGAGCGAGATTCTCCGCGCTGTAGAAGTCACCATTGTTGTGCACGACGACATCATTCCGTG |
| | | GCGTTATCCAGCTAAGCGCGAACTGCAATTTGGAGAATGGCAGCGCAATGACATTCTTGCAGGTATCTTCGAGC |
| | | CAGCCACGATCGACATTGATCTGGCTATCTTGCTGACAAAAGCAAGAGAACATAGCGTTGCCTTGGTAGGTCCA |
| | | GCGGCGGAGGAACTCTTTGATCCGGTTCCTGAACAGGATCTATTTGAGGCGCTAAATGAAACCTTAACGCTATG |
| | | GAACTCGCCGCCCGACTGGGCTGGCGATGAGCGAAATGTAGTGCTTACGTTGTCCCGCATTTGGTACAGCGCAG |
| | | TAACCGGCAAAATCGCGCCGAAGGATGTCGCTGCCGACTGGGCAATGGAGCGCCTGCCGGCCCAGTATCAGCCC |
| | | GTCATACTTGAAGCTAGACAGGCTTATCTTGGACAAGAAGAAGATCGCTTGGCCTCGCGCGCAGATCAGTTGGA |
| | | AGAATTTGTCCACTACGTGAAAGGCGAGATCACCAAGGTAGTCGGCAAA |

### Polynucleotides Encoding and Systems for Expressing Fusion Proteins Comprising a DNA Binding Protein and a Reporter Molecule

The present disclosure provides polynucleotides that encode one or more fusion protein(s), each fusion protein comprising a DNA targeting protein and a reporter molecule. The present disclosure also provides vectors for the expression and delivery of polynucleotides that encode one or more fusion protein(s), each fusion protein comprising a DNA targeting protein and a reporter molecule. Expression and delivery of such polynucleotides may be achieved, for example, by employing a viral vector such as a cocal pseudotyped lentiviral vector, a foamy virus vector, an adenoviral vector, and an adeno-associated viral (AAV) vector. Cocal pseudotyped lentiviral vectors and foamy virus vectors are described in Trobridge et al., Mol Ther 18:725-33 (2008). Adenoviral vectors for use in gene transfer are described in Wang et al., Exp. Hematol. 36:823-31 (2008) and Wang et al., Nat. Med. 17:96-104 (2011).

AAV6-serotype recombinant AAV vectors provide a 4.5 kb payload, sufficient to deliver a fusion protein comprising a DNA binding protein and a reporter molecule. Adenoviral vectors with hybrid capsids are capable of efficiently transducing many types of cells including. Helper-dependent adenoviral vectors offer up to a 30 kb payload, along with transient gene expression, and can be used to deliver multiple DNA binding reporter molecule encoding polynucleotide cassettes.

Integration-deficient lentiviral and foamyviral vectors (IDLV and IDFV) provide 6 kb (IDLV) to 9 kb (IDFV) payloads. High titer stocks may be achieved using a TFF purification step. Vectors with a set of promoter/GFP cassettes may be used to provide efficient and high level expression and may be generated to express individual fusion proteins or combinations of two or more fusion proteins. Multiplex expression permits multiple binding events on a target DNA sequence.

The efficiency of gene targeting, levels of fusion protein expression in individual targeted cells as well as populations of cells and of their progeny may be confirmed in model organisms. Transductions may be followed by single-cell and bulk population assessments of expression of fusion proteins at the RNA and protein levels.

A wide variety of expression systems can be used for expressing the fusion proteins that are disclosed herein. Transient protein production can be used to detect target nucleotide sequence specific binding and corresponding protein-protein interactions between split-reporter proteins in vivo as well as in subcellular localization of the fusion protein complexes.

In such cases, however, protein over-expression may be avoided to, for example, minimize non-specific protein-protein interactions and complex formation. In such cases, the use of weak promoters, low levels of plasmid DNA in during transfection, and plasmid vectors that do not replicate in mammalian cells can be used to express proteins at or near endogenous levels thereby mimicking the physiological cellular environment. Stable cell lines with an expression vector integrated into its genome allows more stable protein expression in the cell population, resulting in more consistent results.

Plasmid vectors for expressing the nucleotide sequences encoding the presently disclosed fusion proteins should be configured to express a fusion protein without disrupting the protein's function. In addition, the expected protein complex must be able to accept stabilization of the fluorescent protein fragment interaction without affecting the protein complex function or the cell being studied. As discussed herein, many fluorescent protein fragments that combine in several ways can be used in generating fusion proteins according to the present disclosure.

Fluorescent protein fragments can associate and fluoresce at low efficiency in the absence of a specific interaction. Therefore, it is important to include controls to ensure that the fluorescence from fluorescent reporter protein reconstitution is not due to nonspecific interactions that are independent from target specific binding. Morell et al., Proteomics 8:3433-3442 (2008). Some controls include fluorophore fragments linked to non-interacting proteins, as the presence of these fusions tend to decrease non-specific complementation and false positive results.

Another control can be created by linking the fluorescent protein fragment to targeting proteins having mutated nucleotide sequence binding domains. So long as the fluorescent fragment is fused to the mutated proteins in the same manner as the wild-type protein, and the protein expression levels and localization are unaffected by the mutation, this serves as a strong negative control, as the mutant proteins, and therefore, the fluorescent fragments, should be unable to interact.

Similarly, the spacing (i.e., number of nucleotides) between a first target nucleotide sequence and a second target nucleotide sequence within a target nucleic acid should be tested empirically to determine the spacing that affords optimal re-association between first and second halves of a split-reporter protein. The present disclosure contemplates that a spacing that is less than optimal will increase steric interference between first and second fusion proteins that are bound to a target sequence. By incrementally increasing the intra-target sequence spacing, an optimal spacing for a given pair of fusion proteins can be determined. Likewise, non-specific interactions between fusion proteins can be controlled by testing variants of the desired target sequences to assess for relative non-specific and/or off-target binding.

Internal controls are also advisable to normalize for differences in transfection efficiencies and protein expression levels in different cells. This can, for example, be accomplished by co-transfecting cells with plasmids that encode the fusion proteins of interest as well as a whole (i.e., not split) reporter protein that fluoresces at a different wavelength from the fluorescent reporter protein. During visualization, the fluorescence intensities of the fusion protein pairs and the internal control which, after subtracting background signal, becomes a ratio that represents the assay efficiency, which can be compared with other ratios to determine the relative efficiencies of the formation of different complexes.

Once the fusion protein pairs and suitable controls have been designed and generated in the appropriate expression system, the plasmids can be transfected into the appropriate cells for protein production and for intracellular characterization. After transfection, a period of between about one to about 24 hours is required to achieve optimal fusion protein production levels and/or optimal interaction of the fusion proteins with its corresponding target sequence and fusion protein pair.

After sufficient time for the fusion protein production, interaction, and fluorescence, the transfected cells can be observed under an inverted fluorescence microscope. Although the fluorescence intensity of complexes is often substantially less than that produced by an intact fluorescent protein, the extremely low auto-fluorescence in the visible range makes the specific signal orders of magnitude higher than the background fluorescence signal. See, Kerppola, Ann. Rev Biophys 37:465-487 (2008).

Detectable fluorescence with fusion protein pairs and an absence of fluorescence with a suitable mutated negative control confirms the specificity of the target specific nucleic acid binding interaction. Non-specific interactions between first and second halves of a split-reporter protein are indicated where the fluorescence intensity is not significantly different between the mutated negative control fusion protein and its wild-type counterpart.

If no fluorescence is detected, an interaction may still exist between the proteins of interest, as the creation of the fusion protein may alter the structure or interaction face of the target protein or the fluorescence fragments may be physically unable to associate. To ensure that this result is not a false negative, that there is no interaction, the protein interaction can be tested in a situation where fluorescence complementation and activation requires an external signal. If the external signal fails to cause fluorescence fragment association, it is likely that the proteins do not interact or there is a physical impediment to fluorescence complementation.

The fusion protein pairs of the present disclosure permit the direct visualization of protein interactions in living cells with limited cell perturbation, and do not rely on secondary effects or staining by exogenous.

The fusion protein pairs of the present disclosure do not require protein complexes to be formed by a large proportion of the proteins or at stoichiometric proportions. The presently disclosed systems can readily detect nucleic acid sequence specific binding interactions, weak interactions, and require only low-level fusion protein production as a consequence of the stability of the split-reporter protein subunits. It is contemplated that re-assembly of a split-reporter protein can be achieved with individual target sequences that are spaced a substantial number of nucleotides apart. The optimal spacing between target sequences will vary on a case to case but it is contemplated that a spacing of at least about 100 nucleotides or about 1000 nucleotides may be adequately detected by the fusion protein pairs disclosed herein. Moreover, the strength of the split-reporter protein interactions can be quantitatively determined by changes in fluorescent signal strength.

It will be understood that the fusion protein pairs disclosed herein may be used to determine and/or assess spatial and temporal changes in fusion protein complex formation as well as in subcellular localization and distribution of nucleotide sequences throughout an individual's body and within a wide range of organ systems.

As discussed herein, linking a fluorescent fragment linkage may alter the folding or structure of the protein of interest, leading to the elimination of an interacting protein's surface binding site. In addition, the arrangement of the fluorescent fragments may prevent fluorophore reconstitution through steric hindrance, although steric hindrance can be reduced or eliminated by using a linker sequence that allows sufficient flexibility for the fluorescent fragments to associate. Therefore, absence of fluorescence complementation may be a false negative and does not necessarily prove that the interaction in question does not occur.

The fusion protein pairs will find use in both in vitro and in vivo applications for the detection of a nucleotide sequence of interest, including a nucleotide sequence within a mammalian cell, such as a disease related cells, a bacterial cell, or a virus. Thus, the presently disclosed fusion proteins can be used for the in vivo imaging of cancer cells within a tumor mass or at sites of cancer metastasis. It is contemplated, therefore, that fusion proteins as disclosed herein may be used in combination with traditional cancer therapies and surgical techniques to detect remaining cancer cells that escaped therapeutic treatment or were not removed by a surgical procedure. As such, fusion proteins may be administered to a human via conventional routes of administration or may be produced following expression from a vector that is administered to the human.

The compositions, systems, and methods described herein can, for example, be used to detect or diagnose a disease or disease state, detect and/or localize the tissue-specific distribution of cancer cells (e.g., metastatic cancer cells that have migrated from the site of origin to secondary sources), identify a pathogen or organism having a known genetic sequence, such as a disease pathogen present within cells of a tissue sample. For example, the presently disclosed compositions, systems, and methods can be used to screen for a bacterial cell within a patient sample, such as a bodily fluid, including nasal or oral fluid, blood, urine, or feces, and wherein the bacterial cell is a staphylococcus and wherein the target nucleic acid is a MecA gene.

The systems disclosed herein can be streamlined by being engineered onto a genechip onto which a bodily fluid sample can be added. The photon output can be read on the chip and can be converted to a simple conclusions such as, for example, “the sample is positive” or “the sample is negative.”

The systems disclosed herein can also be used in methods for the in vivo detection of a disease or for the in vivo treatment of a disease. For example, a light activated toxin can be administered in conjunction with a system, wherein the light activated toxin, which light activated toxin is sensitive to light of the wavelength emitted from a reporter group. When a pair of fusion proteins bind to a disease cell, such as a cancer cell, the functional activity of a reporter molecule is restored, which results in the emission of light at a wavelength and intensity that is sufficient to activate the light activated toxin. The fusion proteins can be administered generally or injected directly into the area of the tumor where it will specifically bind to a tumor-specific nucleotide sequence, thereby causing the reporter molecule to emit light of the appropriate wavelength and activating the light activated toxin. In a similar manner, fusion proteins of the present disclosure can also be administered systemically to a patient, allowed to hoe to a tissue of interest and the resulting signal used to image remaining or metastatic cancer cells, wherein the emitted light is detected to image the remaining cancer cells.

The presently disclosed fusion proteins will also find application in methods for detecting nucleotide sequences within tissue samples or biological fluids. For example, infections disease agents, including viral or bacterial agents, can be detected in in vitro assays on tissue or fluid samples obtained from a patient being tested for such an infectious disease or other disease state that is characterized by the presence of a particular nucleotide sequence in a tissue sample or biological fluid.

Fusion proteins disclosed herein may employ multiple fluorescent proteins having varied fluorescent emission wavelengths. That is, it is contemplated that fusion proteins may be produced that employ a split-reporter from a blue, cyan, green, yellow, red, cherry, and/or Venus fluorescent protein. This range in colors can be exploited in methods wherein two or more target nucleotide sequences are to be assessed, such as the presence of two or more infectious diseases, cancer cells, cell types, etc. Multiple fluorescent protein pairs can also be employed to visualize simultaneously two or more nucleotide sequences within the same cell.

Within certain embodiments, the present disclosure provides systems that comprise a first fusion protein and a second fusion protein, the first fusion protein comprising a first sequence-specific targeting protein in operable combination with a first portion of a split-reporter molecule and the second fusion protein comprising a second sequence-specific targeting protein in operable combination with a second portion of reporter molecule, wherein the first sequence-specific targeting protein binds to a first target nucleotide sequence and the second sequence specific targeting protein binds to a second target nucleic acid sequence and wherein when the first and second nucleotide sequences are in proximity the binding of the first sequence-specific targeting protein to the first target nucleotide sequence and the binding of the second sequence-specific targeting protein to the second nucleotide sequence brings the first portion of the reporter molecule into juxtaposition with the second portion of the reporter molecule thereby restoring the functionality of the reporter molecule such that a signal is emitted and the target nucleic acid can be detected.

Within certain aspects of these embodiments, the first and second fusion proteins comprise first and second sequence specific targeting proteins that are Transcription Activator-like (TAL) effector proteins. Within other aspects of these embodiments, the first and second fusion proteins comprise first and second sequence specific targeting proteins that are homing endonucleases (“HEs”). Within certain aspects of these embodiments, the first and second fusion proteins comprise first and second sequence specific targeting proteins that are three prime repair exonucleases (“TREX”). Within certain aspects of these embodiments, the first and second fusion proteins comprise first and second sequence specific targeting proteins that are zinc finger (“ZF”) proteins.

Within related aspects of these embodiments, the first and second fusion proteins comprise first and second reporter molecules that are selected from split-fluorescent reporter molecules, split-luminescent reporter molecules, Förster resonance energy transfer (FRET) reporter molecules, and Bioluminescence Resonance Energy Transfer (BRET) reporter molecules.

### Methods for Detecting a Target Nucleic Acid

Within other embodiments, the present disclosure provides methods that employ the contacting of a first fusion protein and a second fusion protein to a nucleic acid sample, wherein the first fusion protein comprises a first sequence specific targeting protein in operable combination with a first portion of a split-reporter molecule and the second fusion protein comprises a second sequence specific targeting protein in operable combination with a second portion of a split-reporter molecule, wherein the first sequence specific targeting protein binds to a first target nucleotide sequence and the second sequence specific targeting protein binds to a second target nucleotide sequence and wherein when the first and second nucleotide sequences are both present within the nucleic acid sample are both in proximity, the binding of the first sequence specific targeting protein to the first target nucleotide sequence and the binding of the second sequence specific targeting protein to the second nucleotide sequence brings the first portion of the split-reporter molecule into functional proximity with the second portion of the split-reporter molecule such that the binding of the first and second fusion proteins to the first and second target nucleotide sequences within the nucleic acid sample can be detected.

Within certain aspects of these embodiments, the nucleic acid sample is contacted with first and second fusion proteins, which comprise first and second sequence specific targeting proteins, respectively, that are Transcription Activator-like (TAL) effector proteins. Within certain aspects of these embodiments, the nucleic acid sample is contacted with first and second fusion proteins, which comprise first and second sequence specific targeting proteins, respectively, that are homing endonucleases (“HEs”). Within certain aspects of these embodiments, the nucleic acid sample is contacted with first and second fusion proteins, which comprise first and second sequence specific targeting proteins, respectively, that include a Cas protein, such as a Cas9 protein, and a tracrRNA having specificity for the first and second target nucleotide sequences, respectively. Within certain aspects of these embodiments, the nucleic acid sample is contacted with first and second fusion proteins, which comprise first and second sequence specific targeting proteins, respectively, that are three prime repair exonucleases (“TREX”). Within certain aspects of these embodiments, the nucleic acid sample is contacted with first and second fusion proteins, which comprise first and second sequence specific targeting proteins, respectively, that are zinc finger (“ZF”) proteins.

Within related aspects of these embodiments, the first and second fusion proteins comprise first and second reporter molecules that are selected from split-fluorescent reporter molecules, split-luminescent reporter molecules, Förster resonance energy transfer (FRET) reporter molecules, and Bioluminescence Resonance Energy Transfer (BRET) reporter molecules.

The present disclosure will be best understood in view of the following non-limiting Examples.

### EXAMPLES

### Example 1

### Construction of Fusion Proteins Comprising a Transcription Activator-Like (TAL) Effector DNA Binding Protein and a Reporter Molecule

The Cermak Golden Gate method is employed as follows to generate Transcription Activator-like (TAL) Effector DNA Binding Proteins having target DNA specificity. Separate repeat variable disresidue (RVD) plasmids 1-10 (1. pNI, 2. pNG, etc.) are cloned into a first fusion array plasmid A (pFUS_A). Separate RVD plasmids 11-16 are cloned into a second fusion array plasmid B (pFUS_B). 150 ng each of the fusion and array plasmids are digested and ligated in a single 20 μl reaction and are incubated in a thermocycler for 10, 5 minute cycles at 37° C. and 10 min at 16° C., then heated to 50° C. for 5 min, and 80° C. for 5 min. 1 μl 25 mM ATP and 1 μl DNase is added, the reaction is incubated at 37° C. for 1 h, then transformed into E. coli and the cells are plated onto agar plates.

Individual colonies are used to start overnight cultures. Plasmid DNA is isolated and clones with the correct arrays are identified by restriction enzyme digestion and agarose gel electrophoresis. Intermediary arrays are joined, along with the last RVD the desired context (e.g., Renilla luciferase) using one of the four backbone plasmids. A 20 μl digestion and ligation reaction is prepared as above, but with 150 ng each of the pFUS_A and pFUS_B plasmids containing the intermediary repeat arrays, 150 ng of the backbone plasmid (pTAL3 is used for constructing a TALE monomer) and subjected to thermocycling for 10, 5 minute cycles at 37° C. and 10 min at 16° C., then heated to 50° C. for 5 min, and 80° C. for 5 min. The mixture is incubated at 37° C. for 1 h, then transformed into E. coli and plated onto agar plates. The resulting colonies are used to start overnight cultures.

Plasmid DNA is isolated and clones are identified that contain the final, full-length repeat array (which can be verified by digestion with BstAPI and AatII). Whole new plasmid is ligated into an expression plasmid (containing an origin of replication, an ampicillin resistance marker, and the genetic elements to drive protein expression) and transformed into bacteria. Individual bacterial clones are selected, grown in culture, and expression is induced.

The following three reactions are prepared: (1) TALs plus oligonucleotides having a complete match; (2) TALs plus oligonucleotides having a partial match; and (3) TALs plus oligonucleotides having no match. Fluorescence is measured to ensure that TAL constructs can distinguish between correct sequences.

1-5. (canceled)

6. A fusion protein pair for detecting a target nucleic acid, said fusion protein pair comprising a first fusion protein and a second fusion protein, wherein said first fusion protein comprises a first sequence-specific nucleic acid binding protein that is linked to a first portion of a split-reporter protein and wherein said second fusion protein comprises a second sequence-specific nucleic acid binding protein that is linked to a second portion of a said split-reporter protein.

7. The fusion protein pair of claim 6 wherein said first sequence-specific nucleic acid binding protein specifically binds to a first nucleotide sequence within said target nucleic acid and wherein said second sequence-specific nucleic acid binding protein specifically binds to a second nucleotide sequence within said target nucleic acid.

8. The fusion protein pair of claim 6 wherein said first and said second sequence-specific nucleic acid binding proteins are each independently selected from the group consisting of a Cas9 protein, a transcription activator-like enhancer (“TALE”) protein, a homing endonuclease (“HE”), and a zinc finger (“ZF”) protein.

9. The fusion protein pair of claim 6 wherein said split-reporter molecule is selected from the group consisting of a split-fluorescent reporter molecule, a split-luminescent reporter molecule, a Förster resonance energy transfer (FRET) reporter molecule, and a Bioluminescence Resonance Energy Transfer (BRET) reporter molecule.

10.-13. (canceled)

14. A polynucleotide pair that encodes a fusion protein pair for detecting a target nucleic acid, said polynucleotide pair comprising: (a) a polynucleotide encoding a first fusion protein comprising a first nucleotide sequence that encodes a first sequence-specific nucleic acid binding protein that is linked to a first portion of a split-reporter protein and (b) a polynucleotide encoding a second fusion protein comprising a second nucleotide sequence that encodes a second sequence-specific nucleic acid binding protein that is linked to a second portion of a split-reporter protein.

15. The polynucleotide pair of claim 14 wherein said first sequence-specific nucleic acid binding protein specifically binds to a first nucleotide sequence within said target nucleic acid and wherein said second sequence-specific nucleic acid binding protein specifically binds to a second nucleotide sequence within said target nucleic acid.

16. The polynucleotide pair of claim 14 wherein said first and said second sequence-specific nucleic acid binding proteins are each independently selected from the group consisting of a Cas9 protein, a transcription activator-like enhancer (“TALE”) protein, a homing endonuclease (“HE”), and a zinc finger (“ZF”) protein.

17. The polynucleotide pair of claim 14 wherein said split-reporter molecule is selected from the group consisting of a split-fluorescent reporter molecule, a split-luminescent reporter molecule, a Förster resonance energy transfer (FRET) reporter molecule, and a Bioluminescence Resonance Energy Transfer (BRET) reporter molecule.

18-30. (canceled)

31. A method for detecting a target nucleic acid sequence, said method comprising: contacting a first fusion protein and a second fusion protein to a sample comprising a nucleic acid,
wherein the first fusion protein comprises a first sequence specific nucleic acid binding protein in operable combination with a first portion of a split-reporter molecule and the second fusion protein comprises a second sequence specific nucleic acid binding protein in operable combination with a second portion of the split-reporter molecule,
wherein the first sequence specific nucleic acid binding protein binds to a first target nucleotide sequence and the second sequence specific nucleic acid binding protein binds to a second target nucleotide sequence and
wherein when the first and second nucleotide sequences are both present within the nucleic acid within sample and are both in proximity, the binding of the first sequence specific nucleic acid binding protein to the first target nucleotide sequence and the binding of the second gene-targeting protein to the second target nucleotide sequence brings the first portion of the reporter molecule into juxtaposition with the second portion of the reporter molecule thereby restoring the functionality of the re-assembled split-reporter molecule and facilitating the detection of the target nucleic acid.

32. The method of claim 30 wherein said nucleic acid sample is contacted with first and second fusion proteins, which comprise first and second sequence specific nucleic acid binding proteins, respectively, that are transcription activator-like (TAL) effector proteins.

33. The method of claim 30 wherein said nucleic acid sample is contacted with first and second fusion proteins, which comprise first and second sequence specific nucleic acid binding proteins, respectively, that are homing endonucleases (“HEs”) having specificity for the first and second target nucleotide sequences, respectively.

34. The method of claim 30 wherein said nucleic acid sample is contacted with first and second fusion proteins, which comprise first and second sequence specific nucleic acid binding proteins, respectively, that comprise a Cas protein, such as a Cas9 protein, and a tracrRNA having specificity for the first and second target nucleotide sequences, respectively.

35. The method of claim 30 wherein said nucleic acid sample is contacted with first and second fusion proteins, which comprise first and second sequence specific nucleic acid binding proteins, respectively, that are three prime repair endonucleases (“TREX”) having specificity for the first and second target nucleotide sequences, respectively.

36. The method of claim 30 wherein said nucleic acid sample is contacted with first and second fusion proteins, which comprise first and second sequence specific nucleic acid binding proteins, respectively, that are zinc finger (“ZF”) proteins having specificity for the first and second target nucleotide sequences, respectively.

37. The method of claim 30 wherein said first and second fusion proteins comprise first and second reporter molecules are selected from the group consisting of split-fluorescent reporter molecules, split-luminescent reporter molecules, Förster resonance energy transfer (FRET) reporter molecules, and Bioluminescence Resonance Energy Transfer (BRET) reporter molecules.

38-55. (canceled)

56. The fusion protein pair of claim 6 wherein said split-reporter molecule is selected from the group consisting of a split-Renilla reniformis luciferase protein, a split-Photinus pyralis luciferase protein, and a split-Green Fluorescent protein.

57. The polynucleotide pair of claim 14 wherein said split-reporter molecule is selected from the group consisting of a split-Renilla reniformis luciferase protein, a split-Photinus pyralis luciferase protein, and a split-Green Fluorescent protein.

58. The polynucleotide pair of claim 14 wherein said polynucleotide pair further comprises a vector, which vector is configured to express one or both of said polynucleotide encoding said first fusion protein and said polynucleotide encoding said second fusion protein.

59. The polynucleotide pair of claim 58 wherein said vector is selected from the group consisting of a plasmid vector and a viral vector wherein said viral vector is selected from the group consisting of a cocal vesiculovirus pseudotyped lentiviral vector, a foamy virus vector, an adenoviral vector, and an adeno-associated viral (AAV) vector.

60. The method of claim 30 wherein said split-reporter molecule is selected from the group consisting of a split-Renilla reniformis luciferase protein, a split-Photinus pyralis luciferase protein, and a split-Green Fluorescent protein.
